SEQUENCE LISTING (25 sequences)

SEQ ID NO: 1 - protein, 15 aa, Artificial Sequence
  Description of Artificial Sequence hPD-1.08A light chain CDR1
RASKSVSTSGFSYLH

SEQ ID NO: 2 - protein, 7 aa, Artificial Sequence
  Description of Artificial Sequence hPD-1.08A light chain CDR2
LASNLES

SEQ ID NO: 3 - protein, 9 aa, Artificial Sequence
  Description of Artificial Sequence hPD-1-08A light chain CDR3
QHSWELPLT

SEQ ID NO: 4 - protein, 5 aa, Artificial Sequence
  Description of Artificial Sequence hPD-1.08A heavy chain CDR1
SYYLY

SEQ ID NO: 5 - protein, 17 aa, Artificial Sequence
  Description of Artificial Sequence hPD-1.08A heavy chain CDR2
GVNPSNGGTNFSEKFKS

SEQ ID NO: 6 - protein, 11 aa, Artificial Sequence
  Description of Artificial Sequence hPD-1.08A heavy chain CDR3
RDSNYDGGFDY

SEQ ID NO: 7 - protein, 15 aa, Artificial Sequence
  Description of Artificial Sequence hPD-1.09A light chain CDR1
RASKGVSTSGYSYLH

SEQ ID NO: 8 - protein, 7 aa, Artificial Sequence
  Description of Artificial Sequence hPD-1.09A light chain CDR2
LASYLES

SEQ ID NO: 9 - protein, 9 aa, Artificial Sequence
  Description of Artificial Sequence hPD-1.09A light chain CDR3
QHSRDLPLT

SEQ ID NO: 10 - protein, 5 aa, Artificial Sequence
  Description of Artificial Sequence hPD-1.09A heavy chain CDR1
NYYMY

SEQ ID NO: 11 - protein, 17 aa, Artificial Sequence
  Description of Artificial Sequence hPD-1.09A heavy chain CDR2
GINPSNGGTNFNEKFKN

SEQ ID NO: 12 - protein, 11 aa, Artificial Sequence
  Description of Artificial Sequence hPD-1.09A heavy chain CDR3
RDYRFDMGFDY

SEQ ID NO: 13 - protein, 120 aa, Artificial Sequence [sequence omitted]

SEQ ID NO: 14 - protein, 447 aa, Artificial Sequence
  Description of Artificial Sequence Humanized Antibody Heavy Chain-409A-H heavy chain full length
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 111 aa, Artificial Sequence [sequence omitted]

SEQ ID NO: 16 - protein, 111 aa, Artificial Sequence
  Description of Artificial Sequence Humanized Antibody Light Chain Variable Region-K09A-L-16 light chain variable region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 17 - protein, 111 aa, Artificial Sequence
  Description of Artificial Sequence Humanized Antibody Light Chain Variable Region-K09A-L-17 light chain variable region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 18 - protein, 218 aa, Artificial Sequence
  Description of Artificial Sequence Humanized Antibody Light Chain-K09A-L-11 light chain full length
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 218 aa, Artificial Sequence
  Description of Artificial Sequence Humanized Antibody Light Chain-K09A-L-16 light chain full length
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 218 aa, Artificial Sequence
  Description of Artificial Sequence Humanized Antibody Light Chain-K09A-L-17 light chain full length
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, Artificial Sequence; identical to SEQ ID NO: 14
  Description of Artificial Sequence Humanized Antibody Heavy Chain-Pembrolizumab heavy chain

SEQ ID NO: 22 - protein, 218 aa, Artificial Sequence; identical to SEQ ID NO: 18
  Description of Artificial Sequence Humanized Antibody Light Chain-Pembrolizumab light chain

SEQ ID NO: 23 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 290 aa, Homo sapiens
  mat_peptide (19)..(290)
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2019/0263927 A1
(43) Pub. Date: Aug. 29, 2019

(54) COMBINATION OF A PD-1 ANTAGONIST AND ERIBULIN FOR TREATING UROTHELIAL CANCER

(71) Applicants: Merck Sharp & Dohme Corp., Rahway, NJ (US); Eisai R&D Management Co., Ltd., Tokyo (JP)

(72) Inventor: Martin S. OLIVO, Allendale, NJ (US)

(73) Assignees: Merck Sharp & Dohme Corp., Rahway, NJ (US); Eisai R&D Management Co., Ltd., Tokyo (JP)

(21) Appl. No.: 16/341,579

(22) PCT Filed: Oct. 13, 2017
(86) PCT No.: PCT/US2017/056552

§ 371 Date: Apr. 12, 2019

Related U.S. Application Data
(60) Provisional application No. 62/408,328, filed on Oct. 14, 2016.

(51) Int. Cl. C07K 16/30; A61K 31/351; C07K 16/28; A61P 35/04; A61K 9/00

(52) U.S. Cl. CPC C07K 16/3038; A61K 31/351; A61K 9/0019; A61P 35/04; C07K 16/2896

(57) ABSTRACT

The present disclosure describes combination therapies comprising an antagonist of Programmed Death 1 receptor (PD-1) and eribulin or a pharmaceutically acceptable salt thereof, and the use of the combination therapies for the treatment of urothelial cancer.

### REFERENCE TO RELATED APPLICATIONS

This application claims the benefit of U.S. Provisional Application No. 62/408,328 filed on Oct. 14, 2016, the contents of which are hereby incorporated in their entirety.

### SEQUENCE LISTING

The instant application contains a Sequence listing which has been submitted electronically in ASCII format and is hereby incorporated by reference in its entirety. Said ASCII copy, created on Oct. 12, 2017, is named 213597_0003_00_WO_568866_SL and is 32,656 bytes in size.

### FIELD OF THE INVENTION

The present invention relates to combination therapies useful for the treatment of urothelial cancer (UC). In particular, the invention relates to a combination therapy which comprises an antagonist of a Programmed Death 1 protein (PD-1) and eribulin or a pharmaceutically acceptable salt thereof.

### BACKGROUND OF THE INVENTION

PD-1 is recognized as having a role in immune regulation and the maintenance of peripheral tolerance. PD-1 is moderately expressed on naive T, B, and NKT cells and up-regulated by T/B cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers, and melanoma, it was shown that PD-L1 expression correlated with poor prognosis and reduced overall survival irrespective of subsequent treatment (2-12). Similarly, PD-1 expression on tumor infiltrating lymphocytes was found to mark dysfunctional T cells in breast cancer and melanoma (13-14) and to correlate with poor prognosis in renal cancer (15). Thus, it has been proposed that PD-L1 expressing tumor cells interact with PD-1 expressing T cells to attenuate T cell activation and cancer cell evasion of immune surveillance, thereby contributing to an impaired immune response against the tumor.

Several monoclonal antibodies that inhibit the interaction between PD-1 and one or both of its ligands PD-L1 and PD-L2 are in clinical development for treating cancer. It has been proposed that the efficacy of such antibodies might be enhanced if administered in combination with other approved or experimental cancer therapies, e.g., radiation, surgery, chemotherapeutic agents, targeted therapies, agents that inhibit other signaling pathways that are disregulated in tumors, and other immune enhancing agents.

National Cancer Institute (“NCI”) Protocol No. 7435 is a Phase 1/2 study conducted to evaluate the safety and efficacy of eribulin monotherapy in patients with metastatic UC (mUC). This study was originally designed as a Phase 2 study in patients with advanced UC who had not received any chemotherapy for advanced or recurrent disease. Eribulin monotherapy was shown to provide clinical benefit for treatment of patients with mUC.

### SUMMARY OF THE INVENTION

In one embodiment, the invention provides a method for treating an urothelial cancer, including locally advanced and metastatic transitional cell urothelial cancer as well as for patients who have comorbidity for renal impaiiment making the patient ineligible for certain platinum therapies (e.g., cisplatin, carboplatin, etc.) comprising administering to the individual a combination therapy which comprises a PD-1 antagonist and eribulin or a pharmaceutically acceptable salt thereof (e.g., eribulin mesylate).

In another embodiment, the invention provides a medicament comprising a PD-1 antagonist for use in combination with eribulin or a pharmaceutically acceptable salt thereof (e.g., eribulin mesylate) for treating an urothelial cancer.

In yet another embodiment, the invention provides a medicament comprising eribulin or a pharmaceutically acceptable salt thereof (e.g., eribulin mesylate) for use in combination with a PD-1 antagonist for treating an urothelial cancer.

Other embodiments provide use of a PD-1 antagonist in the manufacture of a medicament for treating an urothelial cancer in an individual when administered in combination with eribulin or a pharmaceutically acceptable salt thereof (e.g., eribulin mesylate) and use of eribulin or a pharmaceutically acceptable salt thereof (e.g., eribulin mesylate) in the manufacture of a medicament for treating an urothelial cancer in an individual when administered in combination with a PD-1 antagonist.

In a still further embodiment, the invention provides use of a PD-1 antagonist and eribulin or a pharmaceutically acceptable salt thereof (e.g., eribulin mesylate) in the manufacture of medicaments for treating an urothelial cancer in an individual. In some embodiments, the medicaments comprise a kit, and the kit can also comprise a package insert comprising instructions for using the PD-1 antagonist in combination with eribulin or a pharmaceutically acceptable salt thereof (e.g., eribulin mesylate) to treat an urothelial cancer in an individual.

In all of the above treatment methods, medicaments, and uses, the PD-1 antagonist inhibits the binding of PD-L1 to PD-1, and preferably also inhibits the binding of PD-L2 to PD-1. In some embodiments of the above treatment methods, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences shown in FIG. 6 (SEQ ID NO: 21 and SEQ ID NO: 22).

In all of the above embodiments of the treatment methods, medicaments, and uses herein, the eribulin is optionally eribulin mesylate.

In some embodiments of the above treatment methods, medicaments, and uses, the individual is a human, and the urothelial cancer is metastatic urothelial cancer or locally advanced urothelial cancer or wherein the patient has comorbidity for renal impairment making the patient ineligible for platinum therapies.

Also, in some embodiments of any of the above treatment methods, medicaments, and uses, the urothelial cancer tests positive for the expression of one or both of PD-L1 and PD-L2. In still other embodiments, the urothelial cancer has elevated PD-L1 expression.

In one embodiment of the above treatment methods, medicaments, and uses, the individual is a human and the cancer is urothelial cancer e.g. that tests positive for human PD-L1.

In another embodiment of the above treatment methods, medicaments, and uses, the urothelial cancer is previously treated with 0, 1, or 2 lines of chemotherapy in the metastatic setting.

Also, in some embodiments of any of the above treatment methods, medicaments, and uses, the antagonist of PD-1 is an antibody or antigen-binding fragment thereof specifically binds to PD-1. In some embodiments, the antibody or antigen binding fragment thereof includes three heavy chain CDRs (CDRH1, CDRH2, and CDRH3) and three light chain CDRs (CDRL1, CDRL2, and CDRL3). In some embodiments, CDRL1 includes the amino acid sequence set forth in SEQ ID NO: 7, CDRL2 includes the amino acid sequence set forth in SEQ ID NO: 8, CDRL3 includes the amino acid sequence set forth in SEQ ID NO: 9, CDRH1 includes the amino acid sequence set forth in SEQ ID NO: 10, CDRH2 includes the amino acid sequence set forth in SEQ ID NO: 11, and/or CDRH3 includes the amino acid sequence set forth in SEQ ID NO: 12.

### BRIEF DESCRIPTION OF THE DRAWINGS

FIG. 1 shows amino acid sequences of the light chain and heavy chain CDRs for an exemplary anti-PD-1 monoclonal antibody useful in the present invention (SEQ ID NOs: 1-6).

FIG. 2 shows amino acid sequences of the light chain and heavy chain CDRs for another exemplary anti-PD-1 monoclonal antibody useful in the present invention (SEQ ID NOs: 7-12).

FIG. 3 shows amino acid sequences of the heavy chain variable region and full length heavy chain for an exemplary anti-PD-1 monoclonal antibody useful in the present invention (SEQ ID NO: 13 and SEQ ID NO: 14).

FIG. 4 shows amino acid sequences of alternative light chain variable regions for an exemplary anti-PD-1 monoclonal antibody useful in the present invention (SEQ ID NOs: 15-17).

FIG. 5 shows amino acid sequences of alternative light chains for an exemplary anti-PD-1 monoclonal antibody useful in the present invention, with FIG. 5A showing the amino acid sequences for the K09A-L-11 and K09A-L-16 light chains (SEQ ID NOs: 18 and 19, respectively) and FIG. 5B showing the amino acid sequence for the K09A-L-17 light chain (SEQ ID NO: 20).

FIG. 6 shows amino acid sequences of the heavy and light chains for pembrolizumab (SEQ ID NOs: 21 and 22, respectively).

FIG. 7 shows amino acid sequences of the heavy and light chains for nivolumab (SEQ ID NOs: 23 and 24, respectively).

FIG. 8 shows a study design of a phase 1b/2, open label, single-arm, multicenter trial.

### DETAILED DESCRIPTION

### I. Abbreviations

Throughout the detailed description and examples of the invention the following abbreviations will be used:

AE Adverse Event

ALT alanine aminotransferase,

ANC Absolute Neutrophil Count

AST aspartate aminotransferase

BOR Best overall response

CDR Complementarity determining region

CHO Chinese hamster ovary

CR Complete Response

DFS Disease free survival

DLT Dose-Limiting Toxicity

DOR Duration of Response

FFPE Formalin-fixed, paraffin-embedded

FR Framework region

IHC Immunohistochemistry or immunohistochemical

irRC Immune related response criteria

mUC metastatic urothelial cancer

NCBI National Center for Biotechnology Information

OR Overall response

OS Overall survival

PD Progressive Disease

PD-1 Programmed Death 1

PD-L1 Programmed Cell Death 1 Ligand 1

PD-L2 Programmed Cell Death 1 Ligand 2

PFS Progression free survival

PP Predictive Probability

PR Partial Response

Q2W One dose every two weeks

Q3W One dose every three weeks

RECIST Response Evaluation Criteria in Solid Tumors

SD Stable Disease

UC Urothelial cancer

VH Immunoglobulin heavy chain variable region

VK Immunoglobulin kappa light chain variable region

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the dosage of a PD-1 antagonist (or eribulin or a pharmaceutically acceptable salt thereof (e.g., eribulin mesylate)), or the length of treatment time with a PD-1 antagonist (or eribulin or a pharmaceutically acceptable salt thereof (e.g., eribulin mesylate))) means that the parameter may vary by as much as 10% above or below the stated numerical value for that parameter.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, and rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full-length monoclonal antibodies), polyclonal antibodies, multispecific antibodies (e.g., bispecific antibodies), humanized and primatized antibodies, fully human antibodies, chimeric antibodies, and camelized single domain antibodies. “Parental antibodies” are antibodies obtained by exposure of an immune system to an antigen prior to modification of the antibodies for an intended use, such as humanization of a parental antibody generated in a mouse for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Human light chains can be classified as kappa and lambda light chains. Furthermore, human heavy chains can be classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, FUNDAMENTAL IMMUNOLOGY Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

The variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32: 1-75; Kabat, et al., (1977) J. Biol. Chem. 252: 6609-6616; Chothia, et al., (1987) J. Mol. Biol. 196: 901-917; or Chothia, et al., (1989) Nature 342: 878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2, and CDRL3 in the light chain variable domain and CDRH1, CDRH2, and CDRH3 in the heavy chain variable domain). See Kabat et al. (1991) SEQUENCES OF PROTEINS OF IMMUNOLOGICAL INTEREST, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)2, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

An “anti-tumor response” when referring to a cancer patient treated with a therapeutic agent, such as a PD-1 antagonist, means at least one positive therapeutic effect, such as a reduced number of cancer cells, a reduced tumor size, a reduced rate of cancer cell infiltration into peripheral organs, a reduced rate of tumor metastasis or tumor growth, or progression free survival. Positive therapeutic effects in cancer can be measured in a number of ways (See e.g., W. A. Weber, J. Null. Med. 50:1S-10S (2009); Eisenhauer et al., supra). In some embodiments, an anti-tumor response to a PD-1 antagonist is assessed using RECIST 1.1 criteria, bidimensional irRC, or unidimensional irRC. In some embodiments, an anti-tumor response is any of SD, PR, CR, PFS, and DFS.

“Bidimensional irRC” refers to the set of criteria described in Wolchok J D, et al., “Guidelines for the evaluation of immune therapy activity in solid tumors: immune-related response criteria,” Clin Cancer Res. 2009; 15(23): 7412-7420. These criteria utilize bidimensional tumor measurements of target lesions, which are obtained by multiplying the longest diameter and the longest perpendicular diameter (cm²) of each lesion.

“Biotherapeutic agent” means a biological molecule, such as an antibody or fusion protein, that blocks ligand/receptor signaling in any biological pathway that supports tumor maintenance and/or growth or suppresses the anti-tumor immune response.

The terms “cancer,” “cancerous,” or “malignant” refer to or describe the physiological condition in mammals that is typically characterized by unregulated cell growth. Examples of cancer include urothelial cancer (e.g., metastatic and/or local advanced UC). Particularly preferred urothelial cancers that may be treated in accordance with the present invention include those characterized by elevated expression of one or both of PD-L1 and PD-L2 in tested tissue samples.

“CDR” or “CDRs” as used herein means complementarity determining region(s) in an immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, epidermal growth factor receptor (EGFR) inhibitors, vascular endothelial growth factor (VEGF) inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Chothia” as used herein means an antibody numbering system described in Al-Lazikani et al., J. Mol. Biol. 273: 927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Diagnostic anti-PD-L monoclonal antibody” means a mAb which specifically binds to the mature form of the designated PD-L (PD-L1 or PD-L2) that is expressed on the surface of certain mammalian cells. A mature PD-L lacks the presecretory leader sequence, also referred to as leader peptide The terms “PD-L” and “mature PD-L” are used interchangeably herein, and mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 25)
MRIFAVFIFMTYWHLLNAFTVTVPKDLYVVEYGSNMTIECKFPVEKQLDL
AALIVYWEMEDKNITQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLENVTSTLRIN
TTTNEIFYCTERRLDPEENHTAELVIPELPLAHPPNERTHLVILGAILLC
LGVALTFIFRLRKGRMMDVKKCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in the copending international patent application PCT/US13/075932, filed 18 Dec. 2013 and published as WO2014/100079 on 26 Jun. 2014. Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P.R. China; Catalog number 10084-R015).

“Dose-limiting toxicity” or “DLT” as used herein means toxicities occurring during the DLT assessment window and considered to be related to pembrolizumab and/or eribulin. Toxicities can include hematologic toxicities (e.g., any Grade 4 thrombocytopenia or neutropenia lasting >7 days) and/or non-hematologic toxicities (e.g., episcleritis, uveitis, or iritis of Grade 2 or higher, Grade 4 toxicity, any Grade 3 toxicity excluding nausea, vomiting, or diarrhea that is controlled by medical intervention within 72 hrs.).

“Duration of Response” is defined as the time from the date that a confirmed objective response is first documented to the date of PD or death due to any cause for those subjects with a confirmed PR or CR.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a polynucleotide alignment algorithm, BLAST®, which is a registered trademark of the National Library of Medicine, wherein the parameters of the algorithm are selected to give the largest match between the respective sequences over the entire length of the respective reference sequences.

The following references relate to BLAST® algorithms often used for sequence analysis: BLAST ALGORITHMS: Altschul, S. F., et al., (1990) J. Mol. Biol. 215: 403-410; Gish, W., et al., (1993) Nature Genet. 3: 266-272; Madden, T. L., et al., (1996) Meth. Enzymol. 266: 131-141; Altschul, S. F., et al., (1997) Nucleic Acids Res. 25: 3389-3402; Zhang, J., et al., (1997) Genome Res. 7: 649-656; Wootton, J. C., et al., (1993) Comput. Chem. 17: 149-163; Hancock, J. M. et al., (1994) Comput. Appl. Biosci. 10: 67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M.O. Dayhoff (ed.), pp. 353-358, Natl. Biomed. Res. Found., Washington, D.C.; Altschul, S. F., (1991) J. Mol. Biol. 219: 555-565; States, D. J., et al., (1991) Methods 3: 66-70; Henikoff, S., et al., (1992) Proc. Natl. Acad. Sci. USA 89: 10915-10919; Altschul, S. F., et al., (1993) J. Mol. Evol. 36: 290-300; ALIGNMENT STATISTICS: Karlin, S., et al., (1990) Proc. Natl. Acad. Sci. USA 87: 2264-2268; Karlin, S., et al., (1993) Proc. Natl. Acad. Sci. USA 90: 5873-5877; Dembo, A., et al., (1994) Ann. Prob. 22: 2022-2039; and Altschul, S. F. “Evaluating the statistical significance of multiple distinct local alignments.” iN THEORETICAL AND COMPUTATIONAL METHODS IN GENOME RESEARCH (S. Suhai, ed.), (1997) pp. 1-14, Plenum, New York.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) SEQUENCES OF PROTEINS OF IMMUNOLOGICAL INTEREST, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116: 731.

“Non-responder patient” when referring to a specific anti-tumor response to treatment with a PD-1 antagonist, means the patient did not exhibit the anti-tumor response to the administered PD-1 antagonist treatment.

“Patient” refers to any single human subject for which therapy is desired or that is participating in a clinical trial, epidemiological study or used as a control.

“PD-1 antagonist” means any chemical compound or biological molecule that blocks binding of PD-L1 expressed on a cancer cell to PD-1 expressed on an immune cell (T cell, B cell, or natural killer T (NKT) cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274, and B7-H for PD-L1; and PDCD1L2, PDL2, B7-DC, Btdc and CD273 for PD-L2. In any of the various aspects and embodiments of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the various aspects and embodiments of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody, or a chimeric antibody, and may include a human constant region. In some embodiments, the human constant region is selected from the group consisting of IgG1, IgG2, IgG3, and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv, and Fv fragments.

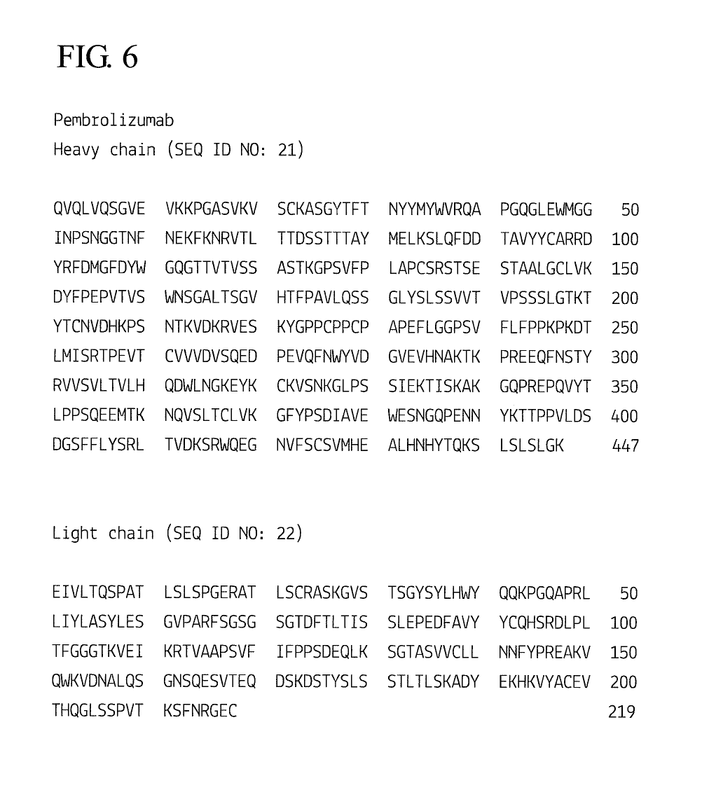

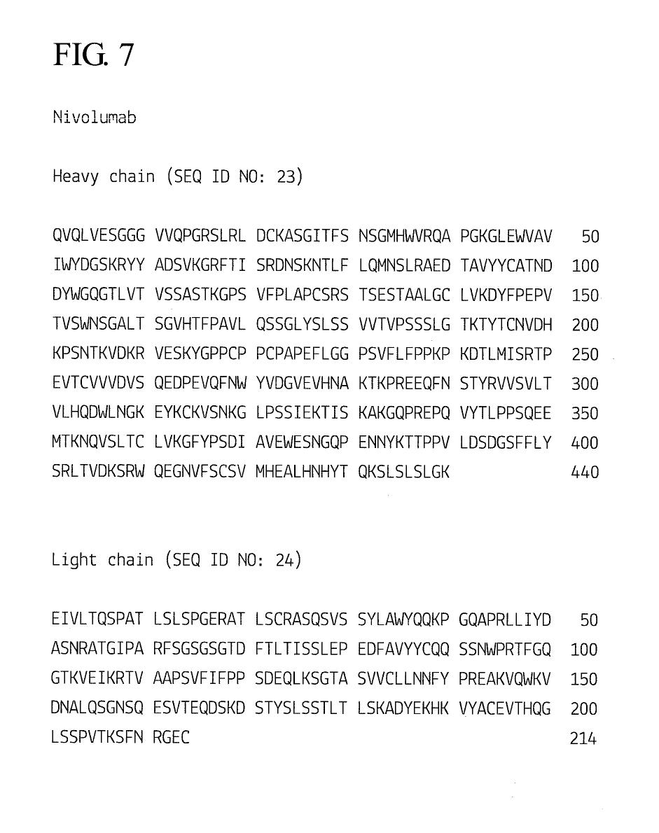

Examples of mAbs that bind to human PD-1, and useful in the treatment methods, medicaments and uses of the present invention, are described in U.S. Pat. Nos. 7,488,802, 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment methods, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in FIG. 6, nivolumab (BMS-936558), a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by MedImmune.

Examples of mAbs that bind to human PD-L1, and useful in any of the various aspects and embodiments of the present invention, are described in WO2013/019906, WO2010/077634 A1 and U.S. Pat. No. 8,383,796. Specific anti-human PD-L1 mAbs useful as the PD-1 antagonist in the various aspects and embodiments of the present invention include MPDL3280A, BMS-936559, MEDI4736, MSB0010718C, and an antibody which comprises the heavy chain and light chain variable regions of SEQ ID NO: 24 and SEQ ID NO: 21, respectively, of WO2013/019906.

Other PD-1 antagonists useful in any of the various aspects and embodiments of the present invention include an immunoadhesion that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fe region of an immunoglobulin molecule. Examples of immunoadhesion molecules that specifically bind to PD-1 are described in WO2010/027827 and WO2011/066342. Specific fusion proteins useful as the PD-1 antagonist in the treatment methods, medicaments and uses of the present invention include AMP-224 (also known as B7-DCIg), which is a PD-L2-FC fusion protein and binds to human PD-1.

In some preferred embodiments of the treatment methods, medicaments and uses of the present invention, the PD-1 antagonist is a monoclonal antibody, or antigen binding fragment thereof, which comprises: (a) light chain CDRs SEQ ID NOs: 1, 2, and 3 and heavy chain CDRs SEQ ID NOs: 4, 5, and 6; or (b) light chain CDRs SEQ ID NOs: 7, 8, and 9 and heavy chain CDRs SEQ ID NOs: 10, 11, and 12.

In other preferred embodiments of the treatment methods, medicaments and uses of the present invention, the PD-1 antagonist is a monoclonal antibody, or antigen binding fragment thereof, which specifically binds to human PD-1 and comprises (a) a heavy chain variable region comprising SEQ ID NO: 13 or a variant thereof, and (b) a light chain variable region comprising an amino acid sequence selected from the group consisting of SEQ ID NO: 15 or a variant thereof; SEQ ID NO: 16 or a variant thereof; and SEQ ID NO: 17 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six, five, four, three, two, or one conservative amino acid substitutions in the framework region. A variant of a light chain variable region sequence is identical to the reference sequence except having up to five conservative amino acid substitutions in the framework region (i.e., outside of the CDRs), and preferably has less than four, three, two or one conservative amino acid substitution in the framework region.

In another preferred embodiment of the treatment methods, medicaments and uses of the present invention, the PD-1 antagonist is a monoclonal antibody which specifically binds to human PD-1 and comprises (a) a heavy chain comprising SEQ ID NO: 14 and (b) a light chain comprising SEQ ID NO: 18, SEQ ID NO: 19, or SEQ ID NO: 20.

In yet another preferred embodiment of the treatment methods, medicaments and uses of the present invention, the PD-1 antagonist is a monoclonal antibody which specifically binds to human PD-1 and comprises (a) a heavy chain comprising SEQ ID NO: 14 and (b) a light chain comprising SEQ ID NO: 18.

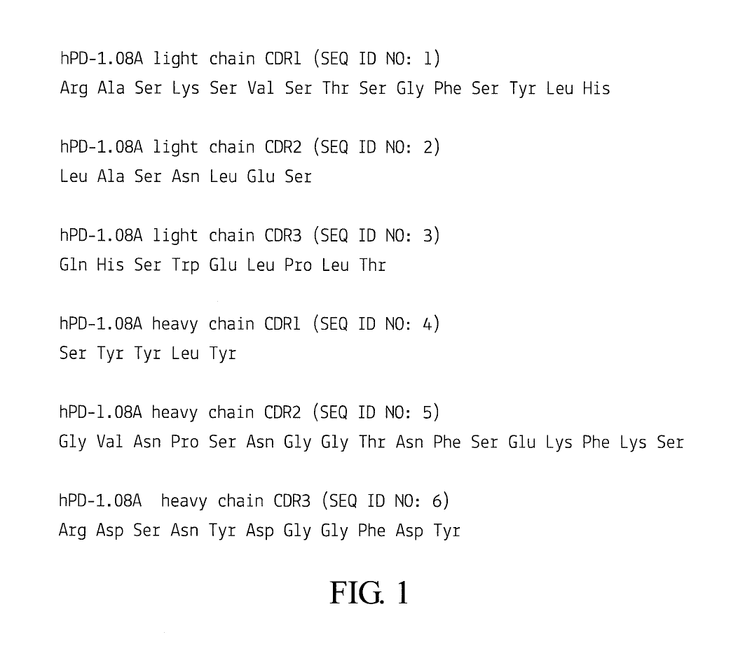

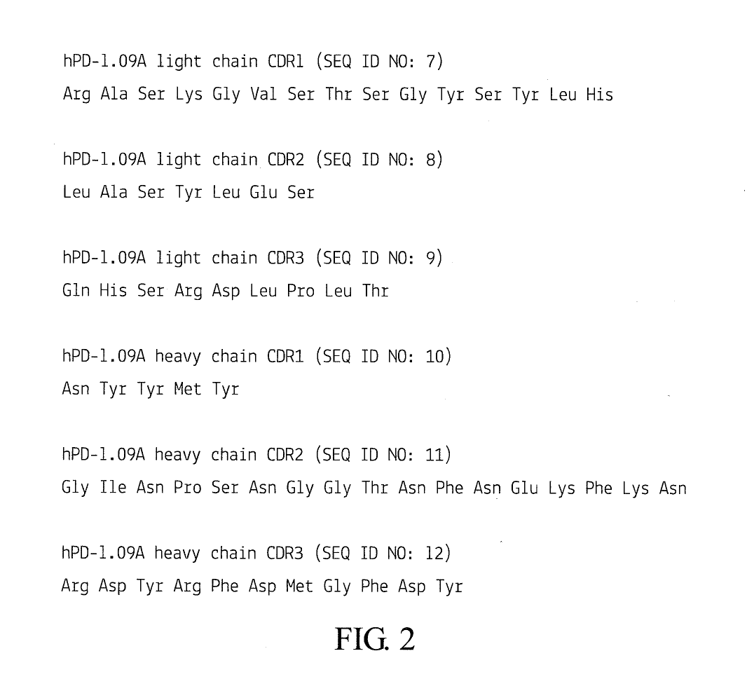

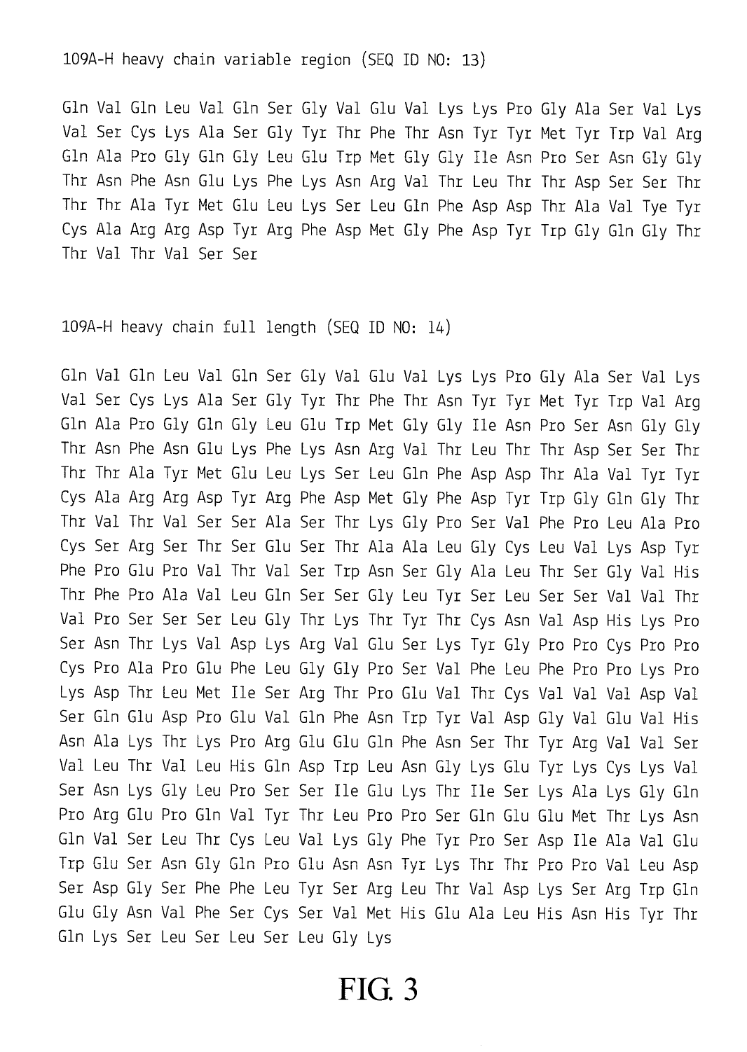

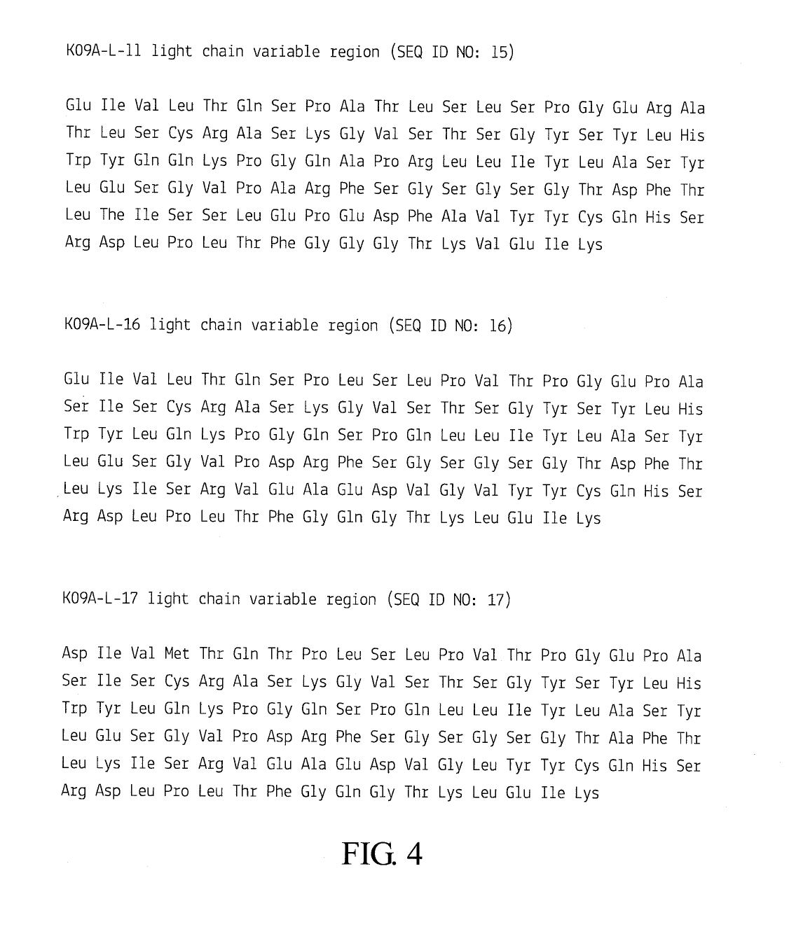

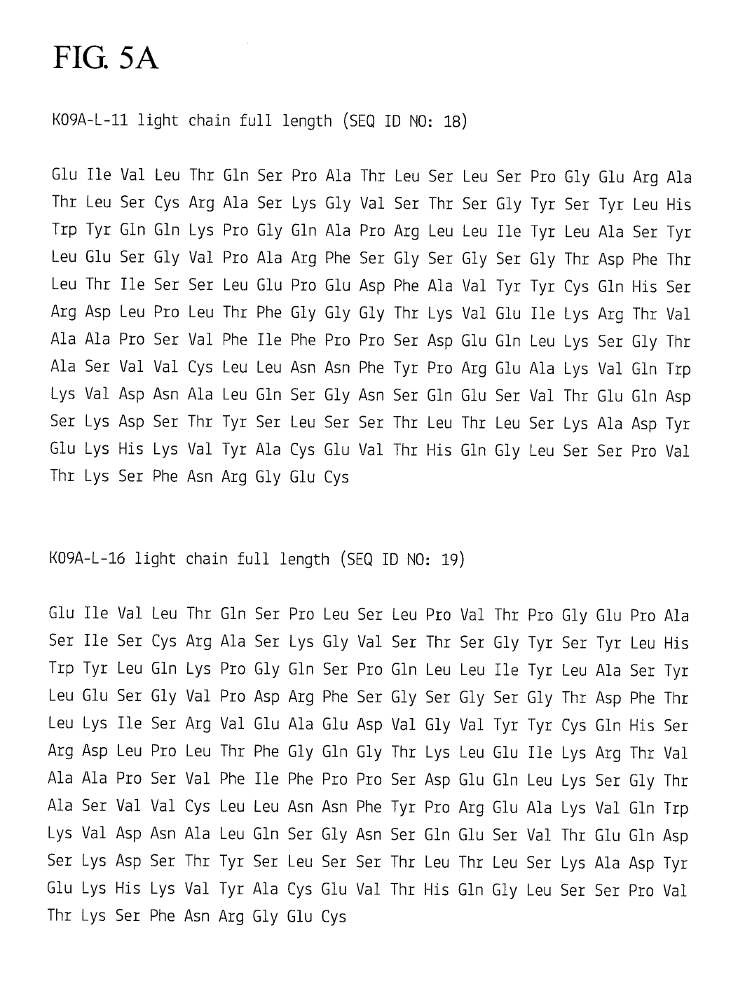

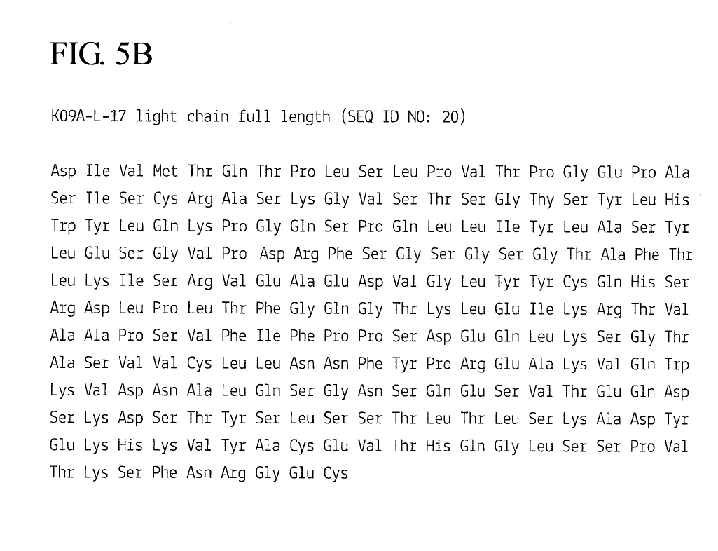

Table 2 below provides a list of the amino acid sequences of exemplary anti-PD-1 mAbs for use in the treatment methods, medicaments, uses of the present invention, and the sequences are shown in FIGS. 1-5.

TABLE 2
EXEMPLARY ANTI-HUMAN PD-1
MONOCLONAL ANTIBODIES
A. Comprises light and heavy chain CDRs of hPD-1.08A in
WO2008/156712

| | |
|---|---|
| CDRL1 | SEQ ID NO: 1 |
| CDRL2 | SEQ ID NO: 2 |
| CDRL3 | SEQ ID NO: 3 |
| CDRH1 | SEQ ID NO: 4 |
| CDRH2 | SEQ ID NO: 5 |
| CDRH3 | SEQ ID NO: 6 |

B. Comprises light and heavy chain CDRs of hPD-1.09A in
WO2008/156712

| | |
|---|---|
| CDRL1 | SEQ ID NO: 7 |
| CDRL2 | SEQ ID NO: 8 |
| CDRL3 | SEQ ID NO: 9 |
| CDRH1 | SEQ ID NO: 10 |
| CDRH2 | SEQ ID NO: 11 |
| CDRH3 | SEQ ID NO: 12 |

C. Comprises the mature h109A heavy chain variable region and one
of the mature K09A light chain variable regions in WO2008/156712

| | |
|---|---|
| Heavy chain VR | SEQ ID NO: 13 |
| Light chain VR | SEQ ID NO: 15, |
| | SEQ ID NO: 16, or |
| | SEQ ID NO: 17 |

D. Comprises the mature 409 heavy chain and one of the mature
K09A light chains in WO2008/156712

| | |
|---|---|
| Heavy chain | SEQ ID NO: 14 |
| Light chain | SEQ ID NO: 18, |
| | SEQ ID NO: 19, or |
| | SEQ ID NO: 20 |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by positron emission tomography (PET) imaging, using a binding agent (e.g., antibody fragment, affibody, and the like) that specifically binds to the desired PD-L target, e.g., PD-L1 or PD-L2. Techniques for detecting and measuring PD-L mRNA expression include RT-PCR and real-time quantitative RT-PCR.

Several approaches have been described for quantifying PD-L1 protein expression in IHC assays of tumor tissue sections. See, e.g., Thompson, R. H., et al., PNAS 101 (49): 17174-17179 (2004); Thompson, R. H. et al., Cancer Res. 66: 3381-3385 (2006); Gadiot, J., et al., Cancer 117: 2192-2201 (2011); Taube, J. M. et al., Sci Transl Med 4: 127ra37 (2012); and Toplian, S. L. et al., New Eng. J Med. 366 (26): 2443-2454 (2012).

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is >5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

The level of PD-L mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

A “pembrolizumab biosimilar” means a biological product manufactured by an entity other than Merck Sharp & Dohme and which is approved by a regulatory agency in any country for marketing as a pembrolizumab biosimilar. In an embodiment, a pembrolizumab biosimilar comprises a pembrolizumab variant as the drug substance. In an embodiment, a pembrolizumab biosimilar has the same amino acid sequence as pembrolizumab.

As used herein, a “pembrolizumab variant” means a monoclonal antibody which comprises heavy chain and light chain sequences that are identical to those in pembrolizumab, except for having three, two, or one conservative amino acid substitutions at positions that are located outside of the light chain CDRs and six, five, four, three, two, or one conservative amino acid substitutions that are located outside of the heavy chain CDRs, e.g., the variant positions are located in the FR regions or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“RECIST 1.1 Response Criteria” as used herein means the definitions set forth in Eisenhauer et al., E. A. et al., Eur. J. Cancer 45: 228-247 (2009) for target lesions or nontarget lesions, as appropriate based on the context in which response is being measured.

“Responder patient” when referring to a specific anti-tumor response to treatment with a combination therapy described herein, means the patient exhibited the anti-tumor response.

“Sample” when referring to a tumor or any other biological material referenced herein, means a sample that has been removed from the subject; thus, none of the testing methods described herein are performed in or on the subject.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5, or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a PD-1 antagonist and eribulin or a pharmaceutically acceptable salt thereof (e.g., eribulin mesylate), or another therapeutic agent to a subject having a cancer, or diagnosed with a cancer, to achieve at least one positive therapeutic effect, such as for example, reduced number of cancer cells, reduced tumor size or tumor burden, reduced rate of cancer cell infiltration into peripheral organs, or reduced rate of tumor metastasis or tumor growth. Positive therapeutic effects in cancer can be measured in a number of ways (see, W. A. Weber, J. Null. Med. 50: 1S-10S (2009); Eisenhauer et al., supra). In some preferred embodiments, response to a PD-1 antagonist is assessed using RECIST 1.1 criteria or irRC. In some embodiments, the treatment achieved by a therapeutically effective amount is any of PR, CR, PFS, DFS, OR, or overall survival (OS). In some preferred embodiments, a gene signature biomarker of the invention predicts whether a subject with a solid tumor is likely to achieve a PR or a CR. The dosage regimen of a therapy described herein that is effective to treat a cancer patient may vary according to factors such as the disease state, age, and weight of the patient, and the ability of the therapy to elicit an anti-cancer response in the subject. While an embodiment of the treatment methods, medicaments and uses of the present invention may not be effective in achieving a positive therapeutic effect in every subject, it should do so in a statistically significant number of subjects as determined by any statistical test known in the art such as the Student's t-test, the chit-test, the U-test according to Mann and Whitney, the Kruskal-Wallis test (H-test), Jonckheere-Terpstra-test, and the Wilcoxon-test.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load,” refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC” refers to the set of criteria described in Nishino M, Giobbie-Hurder A, Gargano M, Suda M, Ramaiya N H, Hodi F S. “Developing a Common Language for Tumor Response to Immunotherapy: Immune-related Response Criteria using Unidimensional measurements.” Clin Cancer Res. 2013; 19(14): 3936-3943). These criteria utilize the longest diameter (cm) of each lesion.

“Variable regions” or “V region” as used herein means the segment of IgG chains for example which is variable in sequence between different antibodies. It extends to Kabat residue 109 in the light chain and 113 in the heavy chain.

“Eribulin” is a synthetic analog of halichondrin B. Eribulin is also known as ER-086526, and has been assigned Chemical Abstract Service (CAS) number 253128-41-5 and US NCI designation number NSC-707389. The mesylate salt of eribulin (eribulin mesylate, which is marketed under the trade name HALAVEN® and is also known as E7389).

The chemical name for eribulin mesylate is 11,15:18,21:24,28-triepoxy-7,9-ethano-12,15-methano-9H,15H-furo [3,2-i]furo [2′,3′: 5,6]pyrano [4,3-b][1,4]dioxacyclopentacosin-5(4H)-one, 2-[(2S)-3-amino-2-hydroxypropyl]hexacosahydro-3-methoxy-26-methyl-20,27-bis(methylene)-, (2R,3R,3aS,7R,8aS,9S,10aR,11S,12R,13aR,13bS,15S,18S,21S,24S,26R,28R,29aS)-methanesulfonate (salt), and it can be depicted as follows:

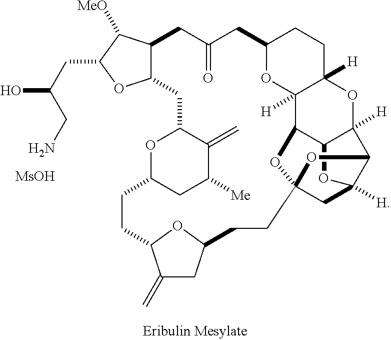

Methods for synthesizing eribulin are described, for example, in U.S. Pat. Nos. 6,214,865; 7,982,060; 8,350,067; and 8,093,410, each of which is incorporated herein by reference.

As noted above, eribulin can optionally be used in the present invention in salt foiins. There are no particular limitations as to the salt used, whether inorganic acid salt or organic acid salt. For example, the salt can be selected from mesylic acid salt (e.g., eribulin mesylate), hydrochloric acid salt, sulfuric acid salt, citrate, hydrobromic acid salt, hydroiodine acid salt, nitric acid salt, bisulfate, phosphoric acid salt, super phosphoric acid salt, isonicotinic acid salt, acetic acid salt, lactic acid salt, salicic acid salt, tartaric acid salt, pantotenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate), and so on. Moreover, it is acceptable to use salt of aluminum, calcium, lithium, magnesium, sodium, zinc, and diethanolamine.

Eribulin is typically provided in liquid form, for intravenous administration to a subject.

### II. Methods, Uses and Medicaments

For metastatic urothelial cancer (mUC), cisplatin-based combination chemotherapy is considered standard first-line treatment. The National Comprehensive Cancer Network (NCCN) Guidelines recommend gemcitabine plus cisplatin (GC) or methotrexate, vinblastine, doxorubicin, and cisplatin (MVAC) as first-line chemotherapy for these patients. An equivalent regimen, evaluated by the European Organisation for Research and the Treatment of Cancer (EORTC), includes paclitaxel, cisplatin, gemcitabine (PCG) (Bellmunt J, von der Maase H, Mead G M, Skoneczna I, De Santis M, Daugaard G, et al., “Randomized phase III Study Comparing paclitaxel/cisplatin/gemcitabine and gemcitabine/cisplatin in patients with locally advanced or metastatic urothelial cancer without prior systemic therapy: EORTC Intergroup Study 30987,” J. Clin. Oncol. 2012; 30(10): 1107-13). However, approximately 30% to 50% of patients are ineligible for cisplatin due to age-associated (and disease-associated) impairment in renal function and performance status (PS) (Galsky M D, Hahn N M, Rosenberg J, Sonparde G, Hudson T, Oh W K, et al., “Treatment of patients with metastatic urothelial cancer “unfit” for cisplatin-based chemotherapy,” 2011; J. Clin. Oncol. 29(17): 2432-8.). Gemcitabine plus carboplatin is the preferred regimen for these patients. There were no US FDA-approved therapies for relapse after platinum-based therapy until May 2016 when Atezolizumab (Tecentriq™, an anti-PD-L1 antibody) was approved in this indication. There remains a significant gap in the determination of viable treatment options for patients with advanced urothelial cancer “unfit” for cisplatin-based chemotherapy or failed on first line of platinum based therapy.

In one aspect of the invention, the invention provides a method for treating an urothelial cancer in an individual comprising administering to the individual a combination therapy which comprises a PD-1 antagonist and eribulin or a pharmaceutically acceptable salt thereof (e.g., eribulin mesylate).

The combination therapy may also comprise one or more additional therapeutic agents. The additional therapeutic agent may be, e.g., a chemotherapeutic other than eribulin or a pharmaceutically acceptable salt thereof (e.g., eribulin mesylate), a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine;

acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammaI1 and calicheamicin phiI1, see, e.g., Agnew, Chem. Intl. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor 9-nitrocamptothecin (RFS 2000); difluoromethylomithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises one or more therapeutic agents and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the eribulin or pharmaceutically acceptable salt thereof (e.g., eribulin mesylate) is administered before administration of the PD-1 antagonist, while in other embodiments, the eribulin or pharmaceutically acceptable salt thereof (e.g., eribulin mesylate) is administered after administration of the PD-1 antagonist.

In some embodiments, at least one of the therapeutic agents in the combination therapy is administered using the same dosage regimen (dose, frequency, and duration of treatment) that is typically employed when the agent is used as monotherapy for treating the same cancer. In other coembodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration.

A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve (e.g. in patients who have comorbidity for renal impairment therefore are ineligible for certain chemotherapeutic agents, such as platinum compounds). In other embodiments, the combination therapy is administered to a patient who failed to achieve a sustained response after prior therapy with a biotherapeutic or chemotherapeutic agent, i.e., that is shown to have disease progression.

A combination therapy of the invention is typically used to treat a tumor that is large enough to be found by palpation or by imaging techniques well known in the art, such as MRI, ultrasound, or computerized axial tomography (CAT) scan.

A combination therapy of the invention is preferably administered to a human patient who has an urothelial cancer that tests positive for PD-L1 expression. In some preferred embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the PD-1 antagonist and eribulin or a pharmaceutically acceptable salt thereof (e.g., eribulin mesylate), but it is envisioned that the physician could order the first or subsequent diagnostic tests at any time after initiation of treatment, such as for example after completion of a treatment cycle.

Selecting a dosage regimen (also referred to herein as an administration regimen) for a combination therapy of the invention depends on several factors, including the serum or tissue turnover rate of the entity, the level of symptoms, the immunogenicity of the entity, and the accessibility of the target cells, tissue, or organ in the individual being treated. Preferably, a dosage regimen maximizes the amount of each therapeutic agent delivered to the patient consistent with an acceptable level of side effects. Accordingly, the dose amount and dosing frequency of each biotherapeutic and chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd., Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348: 601-608; Milgrom et al. (1999) New Engl. J. Med. 341: 1966-1973; Slamon et al. (2001) New Engl. J. Med. 344: 783-792; Beniaminovitz et al. (2000) New Engl. J. Med. 342: 613-619; Ghosh et al. (2003) New Engl. J. Med. 348: 24-32; Lipsky et al. (2000) New Engl. J. Med. 343: 1594-1602; PHYSICIANS' DESK REFERENCE 2003 (Physicians' Desk Reference, 57th Ed.); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents in a combination therapy of the invention may be administered by continuous infusion or by doses at intervals of, e.g., daily, every other day, three times per week, or one time each week, two weeks, three weeks, monthly, bimonthly, etc. A total weekly dose is generally at least 0.05 μg/kg, 0.2_{K}g/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349: 427-434; Herold et al. (2002) New Engl. J. Med. 346: 1692-1698; Liu et al. (1999) J Neurol. Neurosurg. Psych. 67: 451-456; Portielji et al. (2003) Cancer Immunol. Immunother. 52: 133-144.

In some embodiments that employ an anti-human PD-1 mAb as the PD-1 antagonist in the combination therapy, the dosing regimen will comprise administering the anti-human PD-1 mAb at a dose of 1, 2, 3, 5, or 10 mg/kg at intervals of about 14 days (±2 days) or about 21 days (+2 days) or about 30 days (±2 days) throughout the course of treatment.

In other embodiments that employ an anti-human PD-1 mAb as the PD-1 antagonist in the combination therapy, the dosing regimen will comprise administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (+2 days), for doses subsequent to the second dose.

In certain embodiments, a subject will be administered an intravenous (IV) infusion of a medicament comprising any of the PD-1 antagonists described herein.

In one preferred embodiment of the invention, the PD-1 antagonist in the combination therapy is pembrolizumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

In another preferred embodiment of the invention, the PD-1 antagonist in the combination therapy is pembrolizumab, which is administered in a liquid medicament at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, 10 mg Q3W, and flat-dose equivalents of any of these doses, i.e., such as 200 mg Q3W. In some embodiments, pembrolizumab is administered as a liquid medicament which comprises 25 mg/ml pembrolizumab, 7% (w/v) sucrose, 0.02% (w/v) polysorbate 80 in 10 mM histidine buffer pH 5.5, and the selected dose of the medicament is administered by IV infusion over a time period of about 30 minutes.

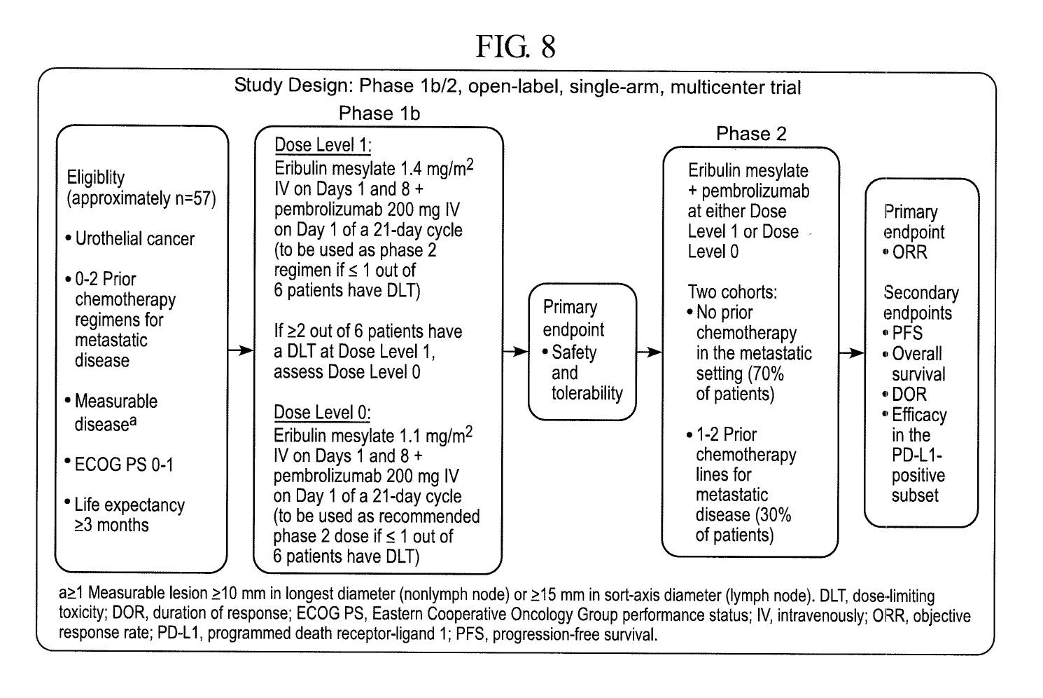

The optimal dose for pembrolizumab in combination with eribulin or a pharmaceutically acceptable salt thereof may be identified by dose escalation of one or both of these agents. In one embodiment, eribulin or a pharmaceutically acceptable salt thereof (e.g., eribulin mesylate) is administered intravenously over about 2-5 minutes at 1.4 mg/m² on days 1 and 8 of a 21-day cycle, and pembrolizumab is administered intravenously over about 30 minutes at 200 mg on day 1 of a 21-day cycle. FIG. 8 shows a study design of a phase 1b/2, open label, single-arm, multicenter trial. A total of approximately 57 adult patients can be enrolled, including six to twelve in the phase 1b and up to 51 in the phase 2 part of the trial. The dose-limiting toxicity (DLT) of the combination regimen of pembrolizumab and eribulin may be determined in the phase 1b part of the trial, which may include a single initial run-in cohort in which at least six patients (up to a maximum of twelve, with up to 50 evaluable patients in total from Phase 1b and Phase 2) may receive eribulin mesylate 1.4 mg/m² (equivalent to 1.23 mg/m² eribulin [expressed as free base]) administered intravenously (IV) on days 1 and 8 and pembrolizumab 200 mg IV on day 1 of a 21-day cycle (Dose Level 1) and will be enrolled in two strata. Stratum 1 includes first-line subjects who are cisplatin ineligible based on renal impairment (creatinine clearance calculated by Cockcroft-Gault method <60 mL/min) and grade 2 hearing loss. Stratum 2 are subjects who have progressed within 12 months of treatment with a platinum containing regimen (cisplatin or carboplatin or novel platinum) in either in the metastatic or perioperative setting. The two strata will have approximately 40% (stratum 1) and 60% (stratum 2), respectively, of the total subjects.

The sample size calculation was based on the assumed ORR of 50% in this study compared to 30% in the historical control. Using a binomial exact test, the power is 0.91 with 50 evaluable subjects to demonstrate statistical significance at a 1-sided alpha of 0.05.

If one or fewer out of six patients has a DLT at Dose Level 1, this regimen may be selected for use in the phase 2 portion of the trial. Otherwise, the eribulin dose may be lowered to 1.1 mg/m² on Days 1 and 8 of the 21-day cycle (Dose Level 0). If one or fewer out of six patients experiences a DLT, the phase 2 portion of the trial may proceed using Dose Level 0 as shown in FIG. 8. In the phase 2 part of the trial, patients may be enrolled in two cohorts according to receipt of prior chemotherapy in the metastatic setting (none vs. 1-2 prior lines). Patients may undergo treatment as long as clinical benefit is demonstrated or until intercurrent illness, unacceptable toxicity, disease progression, withdrawal of consent, or death.

In another embodiment, eribulin or a pharmaceutically acceptable salt thereof (e.g., eribulin mesylate) is administered at 1.4 mg/m² on days 1 and 15 of a 28-day cycle and pembrolizumab is administered intravenously at 200 mg on day 1 of a 21-day cycle.

In another embodiment, if the above-noted dose combinations are not tolerated by the patient, then the dose of eribulin or the pharmaceutically acceptable salt thereof (e.g., eribulin mesylate) is reduced to 1.1 mg/m² on days 1 and 8 of a 21-day cycle (or days 1 and 15 of a 28-day cycle).

In another embodiment, if the above-noted dose combinations are not tolerated by the patient, then the dose of eribulin or the pharmaceutically acceptable salt thereof (e.g., eribulin mesylate) is reduced to 0.7 mg/m² on days 1 and 8 of a 21-day cycle (or days 1 and 15 of a 28-day cycle).

In some embodiments, the patient is selected for treatment with the combination therapy of the invention if the patient has been diagnosed with cancer urothelial cancer (UC), which optionally is metastatic urothelial cancer and/or locally advanced UC.

The present invention also provides a medicament which comprises a PD-1 antagonist as described above and a pharmaceutically acceptable excipient. When the PD-1 antagonist is a biotherapeutic agent, e.g, a mAb, the antagonist may be produced in CHO cells using conventional cell culture and recovery/purification technologies.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some embodiments, a medicament comprising pembrolizumab is provided in a glass vial which contains about 100 mg of pembrolizumab in 4 ml of solution.

The present invention also provides a medicament which comprises eribulin or a pharmaceutically acceptable salt thereof (e.g., eribulin mesylate) and a pharmaceutically acceptable excipient.

The PD-1 antagonist and eribulin or pharmaceutically acceptable salt thereof (e.g., eribulin mesylate) medicaments described herein may be provided as a kit which comprises a first container and a second container and a package insert. The first container contains at least one dose of a medicament comprising a PD-1 antagonist, the second container contains at least one dose of a medicament comprising eribulin or a pharmaceutically acceptable salt thereof (e.g., eribulin mesylate), and the package insert, or label, which comprises instructions for treating a patient for an urothelial cancer using the medicaments. The first and second containers may be comprised of the same or different shape (e.g., vials, syringes, and bottles) and/or material (e.g., plastic or glass). The kit may further comprise other materials that may be useful in administering the medicaments, such as diluents, filters, IV bags and lines, needles and syringes. In some preferred embodiments of the kit, the PD-1 antagonist is an anti-PD-1 antibody and the instructions state that the medicaments are intended for use in treating a patient having an urothelial cancer that tests positive for PD-L1 expression by an IHC assay.

Biomarker Assessments: A blood serum sample for the development of exploratory biomarkers is collected in the mUC cohort during Phase 2 pre-dose on Cycle 1 Day 1, Cycle 1 Day 8, and on Day 1 of all subsequent cycles during Treatment Phase, and at the off-treatment assessment. Blood samples may undergo global proteomic and/or enzyme-linked immunosorbent assay (ELISA)-based analyses or multiplex bead-based immunoassay in an effort to identify protein biomarkers.

A whole blood sample for immune response profiling is collected in the mUC cohort during Phase 2 pre-dose on Cycle 1 Day 1, on Cycle 1 Day 8, and on Day 1 of all subsequent cycles during Treatment Phase, and at the off-treatment assessment. Genomic DNA extracted from blood samples may be used to confirm whether the DNA sequence variants observed in DNA extracted from tumor material are limited to the tumor and to assess the immune response.

Data obtained may be used for research, to assist in developing safer and more effective treatments and will not be used to change the diagnosis of the subject or alter the therapy of the subject. The DNA will not be used to determine or predict risks for diseases that an individual subject does not currently have. Any sample or derivatives (DNA, RNA, and protein) may be stored for up to 15 years to assist in any research scientific questions related to study treatment, cancer and/or for potential diagnostic development.

These and other aspects of the invention, including the exemplary specific embodiments listed below, will be apparent from the teachings contained herein.

### Exemplary Specific Embodiments of the Invention

1. A method for treating an urothelial cancer in an individual comprising administering to the individual a combination therapy which comprises a PD-1 antagonist and eribulin or a pharmaceutically acceptable salt thereof.

2. The method of embodiment 1, wherein the PD-1 antagonist is a monoclonal antibody, or an antigen binding fragment thereof.

3. The method of embodiment 1 or 2, wherein the pharmaceutically acceptable salt of eribulin is eribulin mesylate.

4. A medicament comprising a PD-1 antagonist for use in combination with eribulin or a pharmaceutically acceptable salt thereof for treating an urothelial cancer in an individual, wherein the PD-1 antagonist is a monoclonal antibody, or an antigen binding fragment thereof.

5. A medicament comprising eribulin or a pharmaceutically acceptable salt thereof for use in combination with a PD-1 antagonist for treating an urothelial cancer in an individual.

6. The medicament of embodiment 4 or 5, which further comprises a pharmaceutically acceptable excipient.

7. Use of a PD-1 antagonist in the manufacture of medicament for treating an urothelial cancer in an individual when administered in combination with eribulin or a pharmaceutically acceptable salt thereof.

6. Use of eribulin or a pharmaceutically acceptable salt thereof in the manufacture of a medicament for treating an urothelial cancer in an individual when administered in combination with a PD-1 antagonist.

7. Use of a PD-1 antagonist and eribulin or a pharmaceutically acceptable salt thereof in the manufacture of medicaments for treating an urothelial cancer in an individual.

8. A kit which comprises a first container, a second container and a package insert, wherein the first container comprises at least one dose of a medicament comprising an anti-PD-1 antagonist, the second container comprises at least one dose of a medicament comprising eribulin or a pharmaceutically acceptable salt thereof, and the package insert comprises instructions for treating an individual for urothelial cancer using the medicaments.

9. The kit of embodiment 8, wherein the instructions state that the medicaments are intended for use in treating an individual having urothelial cancer that tests positive for PD-L1 expression by an immunohistochemical (IHC) assay.

10. The method, medicament, use or kit of any of embodiments 1 to 9, wherein the individual is a human and the PD-1 antagonist is a monoclonal antibody, or an antigen binding fragment thereof, which specifically binds to human PD-L1 and blocks the binding of human PD-L1 to human PD-1.

11. The method, medicament, use or kit of embodiment 9, wherein the PD-1 antagonist is MPDL3280A, BMS-936559, MEDI4736, MSB0010718C or a monoclonal antibody which comprises the heavy chain and light chain variable regions of SEQ ID NO: 24 and SEQ ID NO: 21, respectively, of WO2013/019906.

12. The method, medicament, use, or kit of any of embodiments 1 to 9, wherein the individual is a human, and the PD-1 antagonist is a monoclonal antibody, or an antigen binding fragment thereof, which specifically binds to human PD-1 and blocks the binding of human PD-L1 to human PD-1.

13. The method, medicament, use, or kit of embodiment 12, wherein the PD-1 antagonist also blocks binding of human PD-L2 to human PD-1.

14. The method, medicament, use or kit of embodiment 13, wherein the monoclonal antibody, or antigen binding fragment thereof, comprises: (a) light chain CDRs of SEQ ID NOs: 1, 2 and 3 and heavy chain CDRs of SEQ ID NOs: 4, 5, and 6; or (b) light chain CDRs of SEQ ID NOs: 7, 8, and 9 and heavy chain CDRs of SEQ ID NOs: 10, 11, and 12.

15. The method, medicament, use, or kit of embodiment 13, wherein the monoclonal antibody, or antigen binding fragment thereof, comprises light chain CDRs of SEQ ID NOs: 7, 8, and 9 and heavy chain CDRs of SEQ ID NOs: 10, 11, and 12.

16. The method, medicament, use, or kit of embodiment 13, wherein the monoclonal antibody, or antigen binding fragment thereof, comprises a heavy chain variable region of SEQ ID NO: 13 and a light chain variable region of SEQ ID NO: 15.

17. The method, medicament, use, or kit of embodiment 13, wherein the PD-1 antagonist is an anti-PD-1 monoclonal antibody which comprises a heavy chain and a light chain, and wherein the heavy chain comprises SEQ ID NO: 21 and the light chain comprises SEQ ID NO: 22.

18. The method, medicament, use, or kit of embodiment 13, wherein the PD-1 antagonist is an anti-PD-1 monoclonal antibody which comprises a heavy chain and a light chain, and wherein the heavy chain comprises SEQ ID NO: 23 and the light chain comprises SEQ ID NO: 24.

19. The method, medicament, use, or kit of any of embodiments 10-18, wherein the urothelial cancer is a solid tumor.

20. The method, medicament, use, or kit of any of embodiments 10-18, wherein the urothelial cancer is a metastatic urothelial cancer, a locally advanced urothelial cancer, or a urothelial cancer in a patient who has comorbidity for renal impairment.

21. The method, medicament, use, or kit of any of embodiments 10-18, wherein the urothelial cancer is metastatic.

22. The method, medicament, use, or kit of any of embodiments 10-18, wherein the individual has not been previously treated for urothelial cancer.

23. The method, medicament, use or kit of any of embodiments 10-22, the urothelial cancer tests positive for human PD-L1.

24. The method, medicament, use, or kit of embodiment 23, wherein the human PD-L1 expression is elevated in said urothelial cancer.

25. The method, medicament, use or kit of embodiment 13, wherein the PD-1 antagonist is pembrolizumab, a pembrolizumab variant, a pembrolizumab biosimilar or nivolumab.

26. The method, medicament, use, or kit of embodiment 25, wherein pembrolizumab is formulated as a liquid medicament which comprises 25 mg/ml pembrolizumab, 7% (w/v) sucrose, 0.02% (w/v) polysorbate 80 in 10 mM histidine buffer pH 5.5.

27. The method, medicament, use, or kit of any of embodiments 1 to 26, wherein the eribulin is eribulin mesylate.

28. A method for treating a human individual diagnosed with urothelial cancer, comprising administering to the individual a combination therapy which comprises pembrolizumab and eribulin or a pharmaceutically acceptable salt thereof, wherein the eribulin or the pharmaceutically acceptable salt thereof is administered at a dose of 1.4 mg/m², 1.1 mg/m², or 0.7 mg/m² on days 1 and 8 of a 21-day cycle and pembrolizumab is administered at a dose selected from the group consisting of 1 mg/kg Q3W, 2 mg/kg Q3W and 200 mg Q3W.

29. A medicament comprising pembrolizumab for use in combination with eribulin or a pharmaceutically acceptable salt thereof for treating an urothelial cancer in a human individual by a method comprising administering to the individual eribulin or a pharmaceutically acceptable salt thereof at a dose of 1.4 mg/m², 1.1 mg/m², or 0.7 mg/m² on days 1 and 8 of a 21-day cycle and pembrolizumab at a dose selected from the group consisting of 1 mg/kg Q3W, 2 mg/kg Q3W and 200 mg Q3W.

30. A medicament comprising eribulin or a pharmaceutically acceptable salt thereof for use in combination with pembrolizumab for treating an urothelial cancer in a human individual by a method comprising administering to the individual eribulin or a pharmaceutically acceptable salt thereof at a dose of 1.4 mg/m², 1.1 mg/m², or 0.7 mg/m² on days 1 and 8 of a 21-day cycle and pembrolizumab at a dose selected from the group consisting of 1 mg/kg Q3W, 2 mg/kg Q3W, and 200 mg Q3W.

31. The method or medicament of any of embodiments 28 to 31, wherein the urothelial cancer is metastatic and/or locally advanced UC.

32. The method or medicament of embodiment 31, wherein the individual has not been previously treated for urothelial cancer.

33. The method or medicament of any of embodiments 28 to 32, wherein a tissue section of the urothelial cancer removed from the individual prior to administration of the combination therapy tested positive for PD-L1 expression.

34. The method or medicament of embodiment 33, wherein at least 50% of the tumor cells in the tissue section tested positive for PD-L1 expression by an immunohistochemical (IHC) assay.

35. The method or medicament of embodiment 34, wherein the IHC assay employed the antibody 22C3 to detect PD-L1 expression.

36. The method or medicament of any of embodiments 31 to 35, wherein pembrolizumab is administered by IV infusion.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14: 309-314; Barbas (1995) Nature Medicine 1:837-839; Mendez et al. (1997) Nature Genetics 15: 146-156; Hoogenboom and Chames (2000) Immunol. Today 21: 371-377; Barbas et al. (2001) Phage Display: A Laboratory Manual, Cold Spring Harbor

Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163: 5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146: 169-175; Gibellini et al. (1998) J. Immunol. 160: 3891-3898; Hsing and Bishop (1999) J. Immunol. 162: 2804-2811; Everts et al. (2002) J. Immunol. 168: 883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinfbrmatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

Table 3 provides a brief description of the sequences in the sequence listing.

| SEQ ID NO: | Description |
|---|---|
| 1 | hPD-1.08A light chain CDR1 |
| 2 | hPD-1.08A light chain CDR2 |
| 3 | hPD-1-08A light chain CDR3 |
| 4 | hPD-1.08A heavy chain CDR1 |
| 5 | hPD-1.08A heavy chain CDR2 |
| 6 | hPD-1.08A heavy chain CDR3 |
| 7 | hPD-1.09A light chain CDR1 |
| 8 | hPD-1.09A light chain CDR2 |
| 9 | hPD-1.09A light chain CDR3 |
| 10 | hPD-1.09A heavy chain CDR1 |
| 11 | hPD-1.09A heavy chain CDR2 |
| 12 | hPD-1.09A heavy chain CDR3 |
| 13 | 109A-H heavy chain variable region |
| 14 | 409A-H heavy chain full length |
| 15 | K09A-L-11 light chain variable region |
| 16 | K09A-L-16 light chain variable region |
| 17 | K09A-L-17 light chain variable region |
| 18 | K09A-L-11 light chain full length |
| 19 | K09A-L-16 light chain full length |
| 20 | K09A-L-17 light chain full length |
| 21 | Pembrolizumab heavy chain |
| 22 | Pembrolizumab light chain |
| 23 | Nivolumab heavy chain |
| 24 | Nivolumab light chain |
| 25 | Human PD-L1 |

### Example

Study Design

An open-label, single-arm, multicenter, Phase 1b/2 study of eribulin in combination with pembrolizumab in subjects with locally advanced or metastatic transitional cell urothelial cancer (mUC) previously treated with 0 to 2 chemotherapy regimens in the metastatic setting is described below. FIG. 8 shows an example of a study design of a phase 1 b/2, open label, single-arm, multicenter trial. The number of subjects provided for each phase of the study below is a non-limiting example. The particular dosing regimen and/or quantities are also non-limiting. Those of ordinary skill in the art would understand that the number of subjects participating in the study can be increased or decreased. Those of ordinary skill in the art would understand how to modify the dosing regimen and/or quantities for a particular patient or group of subjects.

A subject/patient may be included in the study if the patient has locally advanced or metastatic transitional cell urothelial cancer (mUC) previously treated with 0-2 lines of chemotherapy for metastatic disease. Presence of a measurable disease may be defined as ≥1 lesion of ≥10 mm in long axis diameter for nonlymph nodes or ≥15 mm in short axis diameter for lymph nodes that is serially measurable according to RECIST version 1.1 criteria. The patient may also be required to have adequate bone marrow and liver function, and a life expectancy of ≥3 months. The patient may have resolution of all chemotherapy or radiation-related toxicities to ≤grade 1 severity except for stable sensory neuropathy (grade ≤2) and alopecia (any grade). Additional study inclusion criteria for locally advanced and/or metastatic transitional cell urothelial cancer is the following:
a) Histologically- or cytologically confirmed diagnosis of the renal pelvis, ureter, urinary bladder, or urethra;
b) Previously treated with 0 to 2 lines of systemic anticancer therapy (cytotoxic or targeted anticancer agents) in the metastatic setting;
(c) Subjects who received treatment with a platinum containing regimen (cisplatin or carboplatin or novel platinum) in either in the metastatic or perioperative setting, and have progressed within 12 months of the treatment.

A subject/patient may be excluded from the study if the patient has had previous treatment with eribulin or any anti-PD-1, PD-L1, or PD-L2 agent, or previous participation in a pembrolizumab Merck study. A patient may be excluded if the patient has an autoimmune disease requiring treatment with systemic steroids or immunosuppressant agents, it has been less than 6 months since prior neo/adjuvant chemotherapy, and/or the patient has received treatment with chemotherapy or biological therapy within the previous 3 weeks or radiation or small-molecule targeted therapy within the previous 2 weeks. A patient may be excluded from the study if the patient has a known central nervous system disease, except for those patients with treated brain metastasis who are stable for ≥1 month, having no evidence of progression or hemorrhage after treatment and no ongoing requirement for corticosteroids, as ascertained by clinical examination and brain imaging (magnetic resonance imaging or computed tomography) during the screening period.

Subjects may have received prior neo/adjuvant chemotherapy. The Phase 1b part includes one initial safety run-in cohort in which six to twelve subjects may receive eribulin mesylate 1.4 mg/m² IV on Days 1 and 8 of a 21-day cycle and pembrolizumab 200 mg intravenously (IV) on Day 1 of a 21-day cycle (dose level 1). Eribulin may be infused over about 2-5 minutes per dose and pembrolizumab may be infused over about 25-40 minutes per dose, preferably over about 30 minutes per dose. Eribulin may be diluted in up to 100 mL of 0.9% saline for intravenous infusion.

The Phase 1b part of the study includes one initial safety run-in cohort in which at least 6 subjects may receive eribulin mesylate 1.4 mg/m² intravenously (IV) on Days 1 and 8 of a 21-day cycle and 200 mg pembrolizumab IV on Day 1 of a 21-day cycle (dose level 1). DLT may be assessed in the first cycle. Dose level 1 can be selected as the recommended Phase 2 dose (RP2D) if no more than 1 subject has a DLT. Otherwise, eribulin mesylate dose can be lowered from 1.4 mg/m² to 1.1 mg/m² on Days 1 and 8 of a 21-day cycle (dose level 0). If no more than 1 out of 6 subjects at dose level 0 has a DLT, the Phase 2 part can proceed with dose level 0. Approximately 12 subjects may be enrolled in the Phase 1b part of the study.

In the Phase 1b/2 part, for the mUC cohort: up to 50 evaluable mUC subjects will be enrolled in the study. ORR in the historical control is assumed to be 30%. The ORR in this study is estimated as 50%, which is deemed a clinical meaningful improvement. The null and alternative hypotheses are set as follows: The strata include subjects first-line subjects who are cisplatin ineligible based on renal impairment (creatinine clearance calculated by Cockcroft-Gault method <60 ml/min), grade 2 hearing loss (stratum 1) and subjects who have progressed within 12 months of treatment with a platinum containing regimen (e.g., cisplatin or carboplatin or novel platinum compound) in either in the metastatic or perioperative setting (stratum 2). (Based on the assumption please look at the table). The two strata will have approximately 40% (stratum 1) and 60% (stratum 2) of the total subjects.

It is noted that adequate renal function can be evidenced by: serum creatinine ≤1.5 mg/dL or calculated creatinine clearance ≥20 mL/minute according to the Cockcroft and Gault formula in mUC.

Pharmacokinetic (PK) assessments of eribulin mesylate may be performed in all subjects in the Phase 1b part of the study. Subjects in the Phase 2 part may undergo sparse PK sampling for population pharmacokinetic/pharmacodynamic (PK/PD) analysis where feasible.

Study Treatments.

Combination doses can be investigated in one cohort for mUC. Eribulin mesylate 1.4 mg/m² via IV injection over 2 to 5 minutes administered on Day 1 and Day 8 and 200 mg pembrolizumab via IV infusion over 30 minutes administered on Day 1 (21-day cycle). Alternative doses may be explored to identify the RP2D prior to the start of the Phase 2 part if necessary. The eribulin mesylate dose may be reduced/delayed; the pembrolizumab dose may be delayed per protocol in the event of toxicity. Dose delays and modifications for toxicities associated with eribulin mesylate and pembrolizumab are described in detail below.

### Duration of Treatment.

Subjects may continue to receive study treatment until confirmed disease progression, development of unacceptable toxicity, subject request, withdrawal of consent, or study termination by the sponsor.

Subjects may be permitted to continue study treatment beyond RECIST 1.1-defined disease progression as long as the treating investigator considers that there is clinical benefit and, the subject is tolerating study treatment. The assessment of clinical benefit should take into account whether the subject is clinically deteriorating and unlikely to receive further benefit from continued treatment. Subjects may discontinue study treatment upon evidence of further progression and/or loss of clinical benefit, as judged by the Investigator.

### Efficacy Analyses

Primary Efficacy Phase 1b.

The study can include at least one safety run-in cohort in which six metastatic urothelial cancer patients who receive eribulin mesylate 1.4 mg/m² on Days 1 and 8 and 200 mg pembrolizumab on Day 1 of a 21-day cycle (dose level 1). Subjects may be observed for dose-limiting toxicity in the first cycle. The purpose of the safety run-in cohort(s) is to study safety of the 2-drug combination. The Phase 2 part may proceed with dose level 1 when no more than one subject has a DLT. Otherwise, a lower eribulin mesylate dose of 1.1 mg/m² and 200 mg pembrolizumab can be evaluated in another cohort of six subjects (dose level 0). If no more than 1 subject has a DLT, the Phase 2 part will proceed with dose level 0 as the RP2D. Otherwise, alternative doses (eribulin mesylate 0.7 mg/m²) may be explored prior to the start of the Phase 2 part.

Primary Efficacy Phase 2.

Bayesian predictive probability (PP) can be used to monitor the response rate after post baseline tumor assessments of at least 38 subjects are available. The calculation of PP is based on the goal of claiming superiority of the combination at the end of the study if

P(p>0.2|data)≥0.95 (Eqn. 1)

where p is the response rate of the combination, 0.2 is the response rate of historical control, based on single-agent pembrolizumab and eribulin mesylate in recent trials; 0.95 is the prespecified target probability (θ_{T}) and P(p>0.2|data) is the posterior probability. On the basis of the accumulated data so far in the study, the probabilities of all possible future outcomes that lead to equation (1) at the end of the study may be added in order to obtain the predictive probability of the efficacy of the combination. Therefore, early determination is possible for claiming the combination efficacious when PP is above a prespecified upper threshold (θ_{U}) or for claiming futility when PP is below a prespecified lower threshold (θ_{L}). The upper and lower cutoff probabilities for decision-making, θ_{U} and θ_{L}, are set as 0.99 and 0.025. Under the predictive monitoring, the study can proceed as follows:

If PP>θ_{U}(=0.99), stop the study and claim the combination efficacious or promising;

If PP<θ_{L}(=0.025), stop the study and claim the combination not promising;

Otherwise, continue the study until the number of evaluable subjects reaches to 80.

The Bayesian stopping boundaries are included in Table 4 below. During the study, PP may be calculated with updated response information until the boundary is crossed. In case continuous PP monitoring is not conducted because of operational and logistic reasons (e.g., delayed tumor assessments and fast enrollment) or it is decided to take the study to full enrollment in order to gather more efficacy data, posterior probability in equation (1) may be evaluated to determine the efficacy of the combination regimen after the tumor response status has been collected from the last evaluable subjects. That is, to claim efficacy if P (p>0.2|data) ≥0.95. A 2-sided 95% credible interval of objective response rate in the evaluable subjects may be constructed to aid the interpretation of the results.

TABLE 4
Bayesian stopping boundaries

| N | LB | UB |
|---|---|---|
| 38 | 6 | 16 |
| 39 | 7 | 16 |
| 40 | 7 | 17 |
| 41 | 7 | 17 |
| 42 | 8 | 17 |
| 43 | 8 | 17 |
| 44 | 8 | 18 |
| 45 | 8 | 18 |
| 46 | 9 | 18 |
| 47 | 9 | 18 |
| 48 | 9 | 19 |
| 49 | 10 | 19 |
| 50 | 10 | 19 |
| 51 | 10 | 19 |
| 52 | 10 | 20 |
| 53 | 11 | 20 |
| 54 | 11 | 20 |
| 55 | 11 | 20 |
| 56 | 12 | 20 |
| 57 | 12 | 21 |
| 58 | 12 | 21 |
| 59 | 13 | 21 |
| 60 | 13 | 21 |
| 61 | 13 | 21 |
| 62 | 14 | 22 |
| 63 | 14 | 22 |
| 64 | 14 | 22 |
| 65 | 15 | 22 |
| 66 | 15 | 22 |
| 67 | 15 | 22 |
| 68 | 16 | 23 |
| 69 | 16 | 23 |
| 70 | 17 | 23 |
| 71 | 17 | 23 |
| 72 | 17 | 23 |
| 73 | 18 | 23 |
| 74 | 18 | 23 |
| 75 | 19 | 23 |
| 76 | 19 | 23 |
| 77 | 20 | 23 |
| 78 | 20 | 23 |
| 79 | 21 | 23 |
| 80 | 22 | 23 |

N = number
LB = lower bound
UB = upper bound

Tumor assessment can be performed based on, for example, RECIST 1.1 and irRC. Efficacy can be evaluated by objective tumor responses provided by the investigator according to Response Evaluation Criteria in Solid Tumors (RECIST [version 1.1]) for use in the analysis of primary endpoint (ORR), secondary endpoints (PFS and DOR), and an exploratory endpoint (clinical benefit rate (CBR)). Tumor assessments can be performed every 9 weeks+1 week using consistent imaging methodology (i.e., CT scan/MRI or bone scan) and consistent use or nonuse of contrast media). In the exploratory analyses, clinical activity for the combination treatment including ORR, PFS, DOR, and CBR may also be evaluated using the irRC. In addition, OS status (disposition) may be assessed throughout the study.

Primary Efficacy Final Analysis. While it is possible to determine the efficacy of the combination regimen in terms of ORR early in the study to aid quick decision-making, the final analysis may be performed after all ongoing subjects complete at least 24 weeks of treatment, or discontinue from treatment, and at least 75% subjects have disease progression or death event. Primary, secondary, and exploratory endpoints may be summarized overall and by cohorts. Subjects in the Phase 1b part who were treated at the phase 2 dosing regimen and who were deemed evaluable may be combined with Phase 2 subjects in the efficacy analysis.

Secondary Efficacy. Progression-free survival (PFS), OS, and DOR can be analyzed using Kaplan-Meier product-limit estimates. Median PFS and OS and the cumulative probability of PFS, OS, and DOR at 6 and 12 months can be presented with 2-sided 95% confidence intervals (Cis) if estimable. The cumulative PFS, OS, and DOR can be plotted over time. The median and first and third quartiles from Kaplan-Meier estimation for PFS, OS, and DOR may be provided with 95% CIs if estimable. The primary and secondary efficacy endpoints (i.e., ORR, PFS, OS, and DOR) can be further evaluated in the PD-L1 Positive Set after a cutoff point is determined with external data. The clinical utility of PD-L1 as a predictive marker in mUC subjects who receive eribulin and pembrolizumab combination treatment can be assessed.

Treatments Administered.

Eribulin mesylate and pembrolizumab can be administered as described in Table 5.

TABLE 5
Treatments administered

| | | | | Infusion | |
|---|---|---|---|---|---|
| Drug Name | Dose | | Dose Form | Rate | Day/Cycle |
| Eribulin | 1.4 | mg/m² | IV infusion | Infused over | Day 1 and |
| Mesylate | | | | 2-5 minutes | Day 8 of |
| | | | | | each 21- |
| | | | | | day cycle |
| Pembrolizumab | 200 | mg | IV infusion | Infused over | Day 1 of |
| | | | | 30 minutes | each 21- |
| | | | | (−5 min/+10 | day cycle |
| | | | | min range | |
| | | | | allowed) | |

The amount of eribulin mesylate (as calculated above) can be withdrawn from the appropriate number of vials into a syringe. This may be administered directly as an intravenous injection over 2 to 5 minutes or diluted in up to 100 mL 0.9% saline for IV infusion over 2 to 5 minutes. No special tubing is required for IV administration of eribulin mesylate.

Pembrolizumab may be administered up to 3 days before or after the scheduled Day 1 of each cycle. When the 2 study drugs are scheduled to be administrated simultaneously on Day 1 of each 21-day cycle, pembrolizumab can be given first and followed by eribulin mesylate. Subjects initially treated with eribulin mesylate and pembrolizumab can remain on one or both study drugs in the presence of clinical benefit until intercurrent illness, unacceptable toxicity, or disease progression occurs, or until the subject withdraws consent. In the event of an AE leading to treatment interruption or delay of either study drug, the subject may continue treatment with the other study drug, as long as there is a clinical benefit.

Eribulin mesylate dose may be reduced/delayed during the study. Dose interruption and dose reduction instructions for subjects who experience eribulin toxicity are presented in Table 6:

TABLE 6
Eribulin Mesylate Dose Adjustments for Toxicity

| | Eribulin |
|---|---|
| | Mesylate Dose |
| Adverse Reaction/Toxicity^{a} | Modification |
| Hematologic: | |
| ANC <500 cells/mm³ lasting >7 days in the previous | 1.1 mg/m² |
| cycle despite use of growth factors, recovered to | |
| Grade ≤2 | |
| ANC <1,000 cells/mm³ complicated by fever or | 1.1 mg/m² |
| infection despite use of growth factors, recovered to | |
| Grade <2 | |
| Nonhematologic^{b}: | |
| Grade 3 or 4 event in the previous cycle, recovered to | 1.1 mg/m² |
| Grade <2 | |
| Recurrence of any Grade 3 or 4 event despite reduction | 0.7 mg/m² |
| to 1.1 mg/m² | |
| Recurrence of any Grade 3 or 4 event despite reduction | Consider |
| to 0.7 mg/m² | discontinuation |
| | of combination |
| | treatment |

ANC = absolute neutrophil count.
^{a}Toxicities graded in accordance with National Cancer Institute Common Terminology Criteria for Adverse Events, Version 4.03 (NCI-CTCAE, v 4.03).
^{b}Eribulin mesylate related.

As an example of dose delay, eribulin may not be administered in the event of any of the following: (1) absolute neutrophil count (“ANC”)<1000, (2) platelets <75,000/mm³, and/or (3) Grade 3 or 4 nonhematological toxicities. Eribulin Day 8 dose may be delayed for a maximum of up to 7 additional days (total of 15 days). If toxicities do not resolve or improve to ≤Grade 2 severity by Day 15, the Eribulin Day 8 dose may be omitted and the next cycle may not be initiated until at least two weeks later than the Day 8. If a dose is delayed for toxicities wherein the patient later recovered to Grade 2 severity or less, then administration of eribulin may resume at a reduced dose as set out in the above table.

Pembrolizumab may not be subject to dose reductions. The dose of pembrolizumab may be delayed because of an adverse event. Adverse events, both nonserious and serious, associated with pembrolizumab exposure may represent an immunologic etiology. These adverse events may occur shortly after the first dose or several months after the last dose or treatment. For drug-related toxicities and severe or life-threatening AEs that occur soon after a pembrolizumab dose, pembrolizumab must be withheld or discontinued per Table 7:

TABLE 7
Dose Modification Guidelines for Pembrolizumab-Related Adverse Events

| | Hold | | |
|---|---|---|---|
| | Treatment | Timing for Restarting | Treatment |
| Toxicity | For Grade | Treatment | Discontinuation |
| Diarrhea/Colitis | 2-3 | Toxicity resolves to Grade | Toxicity does not resolve |
| | | 0-1. | within 12 weeks of last |
| | | | dose or inability to reduce |
| | | | corticosteroid to 10 mg or |
| | | | less of prednisone or |
| | | | equivalent per day within |
| | | | 12 weeks. |
| | 4 | Permanently discontinue. | Permanently discontinue. |
| AST, ALT, or | 2 | Toxicity resolves to Grade | Toxicity does not resolve |
| Increased | | 0-1. | within 12 weeks of last |
| Bilirubin | | | dose. |
| | 3-4 | Permanently discontinue | Permanently discontinue. |
| | | (see exception below).^{a} | |
| Type 1 diabetes | T1DM or | Hold pembrolizumab for | Resume pembrolizumab |
| mellitus (if new | 3-4 | new onset Type 1 diabetes | when subjects are clinically |
| onset) or | | mellitus or Grade 3-4 | and metabolically stable. |
| Hyperglycemia | | hyperglycemia associated | |
| | | with evidence of beta cell | |
| | | failure. | |
| Hypophysitis | 2-4 | Toxicity resolves to Grade | Toxicity does not resolve |
| | | 0-1. Treatment with | within 12 weeks of last |
| | | pembrolizumab can be | dose or inability to reduce |
| | | continued while endocrine | corticosteroid to 10 mg or |
| | | replacement therapy is | less of prednisone or |
| | | instituted. | equivalent per day within |
| | | | 12 weeks. |
| Hyperthyroidism | 3 | Toxicity resolves to Grade | Toxicity does not resolve |
| | | 0-1. | within 12 weeks of last |
| | | | dose or inability to reduce |
| | | | corticosteroid to 10 mg or |
| | | | less of prednisone or |
| | | | equivalent per day within |
| | | | 12 weeks. |
| | 4 | Permanently discontinue. | Permanently discontinue. |
| Hypothyroidism | | Treatment with | Treatment with |
| | | pembrolizumab can be | pembrolizumab can be |
| | | continued while thyroid | continued while thyroid |
| | | replacement therapy is | replacement therapy is |
| | | instituted. | instituted. |
| Infusion Reaction | 3-4 | Permanently discontinue. | Permanently discontinue. |
| Pneumonitis | 2 | Toxicity resolves to Grade | Toxicity does not resolve |
| | | 0-1. | within 12 weeks of last |
| | | | dose or inability to reduce |
| | | | corticosteroid to 10 mg or |
| | | | less of prednisone or |
| | | | equivalent per day within |
| | | | 12 weeks. |
| | 3-4 | Permanently discontinue. | Permanently discontinue. |
| Renal Failure or | 2 | Toxicity resolves to Grade | Toxicity does not resolve |
| Nephritis | | 0-1 | within 12 weeks of last |
| | | | dose or inability to reduce |
| | | | corticosteroid to 10 mg or |
| | | | less of prednisone or |
| | | | equivalent per day within |
| | | | 12 weeks. |
| | 3-4 | Permanently discontinue. | Permanently discontinue. |
| All Other Drug- | 3 or Severe | Toxicity resolves to Grade | Toxicity does not resolve |
| Related Toxicity^{b} | | 0-1. | within 12 weeks of last |
| | | | dose or inability to reduce |
| | | | corticosteroid to 10 mg or |
| | | | less of prednisone or |
| | | | equivalent per day within |
| | | | 12 weeks. |
| | 4 | Permanently discontinue. | Permanently discontinue. |

AE = adverse event,
ALT = alanine aminotransferase,
AST = aspartate aminotransferase
Note:
Permanently discontinue for any severe or Grade 3 drug-related adverse event (AE) that recurs or for any life-threatening event.
^{a}For subjects with liver metastasis who begin treatment with Grade 2 AST or ALT, if AST or ALT increases by greater than or equal to 50% relative to baseline and lasts for at least 1 week, then subjects should be discontinued.
^{b}Subjects with an intolerable or persistent Grade 2 drug-related AE may have study drug held at the physician's discretion. Permanently discontinue study drug for persistent Grade 2 adverse reactions (except for alopecia and peripheral sensory neuropathy for which treatment with study drug has been held) that do not recover to Grade 0-1 within 12 weeks of the last dose.

Treatment/End of Treatment Assessments

Dose-limiting toxicity (DLT) assessments can be conducted for Phase 1b subjects. DLTs include Hematologic Toxicities such as any Grade 4 thrombocytopenia or neutropenia lasting >7 days, and Nonhematologic Toxicities including:
Episcleritis, uveitis, or iritis of Grade 2 or higher
Any Grade 4 toxicity
Any Grade 3 toxicity EXCLUDING:
   Nausea/vomiting/diarrhea controlled by medical intervention within 72 hours
   Grade 3 rash in the absence of desquamation, no mucosal involvement, does not require steroids, and resolves to Grade 1 by the next scheduled dose of pembrolizumab
   Transient Grade 3 AST or ALT elevation, defined as no more than 3 days with or without steroid use
Discontinuation or delay of more than 2 weeks of either study medication due to treatment-related AE can be considered as a DLT

Subjects enrolled in Phase 1b can be assessed for DLTs during a DLT assessment window of the first cycle of 21 days. Subjects who discontinue study treatment prior to completing the DLT assessment window for any reason other than a DLT may be replaced.

Safety assessments can include monitoring and recording of adverse events (AE) throughout the study, including Common Terminology Criteria for Adverse Events v4.03 grades for both increasing and decreasing severity and serious adverse events; regular monitoring of hematology, clinical chemistry, and urine; periodic measurement of vital signs; and performance of physical examinations. Physical examination and laboratory evaluation for hematology can be performed at baseline (day 1) and day 8 of each treatment cycle, and within 30 days of the final treatment. Laboratory evaluation for chemistry can be performed at baseline (day 1) and within 30 days of the final treatment. Thyroid function will be assessed at the screening visit and then every 2 cycles throughout the study.

Study Endpoints

Primary Endpoint.

The primary endpoints are safety and tolerability (based on RECIST Version 1.1) for the phase 1b part of the study and objective response rate for the phase 2 part of the study (ORR). ORR is defined as the proportion of subjects who had a BOR of CR or PR by RECIST 1.1.

Secondary Endpoints.

The secondary endpoints of the study are progression-free survival, overall survival, duration of response, and efficacy in a subset of patients defined by PD-L1 expression.
Progression-Free Survival (PFS)—defined as the time from date of first dose of study drug to date of first documentation of disease progression or death, whichever occurs first
Overall Survival (OS)—defined as the time from the date of first dose of study drug until date of death from any cause. Subjects who are lost to follow-up and those who are alive at the date of data cut-off will be censored at the date the subject was last known alive, or date of data cut-off, whichever occurs first.
Duration of Response (DOR)—defined as the time from the date that a confirmed objective response is first documented to the date of progressive disease (“PD”) or death due to any cause for those subjects with a confirmed PR or CR.

Exploratory Endpoints:
Clinical Benefit Rate (CBR)—defined as the proportion of subjects who had BOR of CR, PR, or durable stable disease (SD) (≥24 weeks)
Disease Control Rate (DCR)—defined as the proportion of subjects who had BOR of CR, PR or SD
Durable SD Rate—defined as proportion of subjects who have the duration of SD ≥24 weeks after randomization
Pharmacokinetics (PK) of eribulin mesylate will be assessed for identifying potential effects of pembrolizumab co-administration ORR, PFS, DOR, and CBR using irRECISTAll above endpoints based on tumor measurement will be assessed according to RECIST 1.1, unless otherwise specified.

### REFERENCES

1. Sharpe, A. H, Wherry, E. J., Ahmed R., and Freeman G. J., The function of programmed cell death 1 and its ligands in regulating autoimmunity and infection. Nature Immunology (2007); 8: 239-245.
2. Dong H et al., Tumor-associated B7-H1 promotes T-cell apoptosis: a potential mechanism of immune evasion. Nat Med. 2002 August; 8(8): 793-800.
3. Ghebeh et al., The B7-H1 (PD-L1) T lymphocyte-inhibitory molecule is expressed in breast cancer patients with infiltrating ductal carcinoma: correlation with important high-risk propgnostic factors. Neoplasia (2006) 8: 190-198.
4. Hamanishi J et al., Programmed cell death 1 ligand 1 and tumor-infiltrating CD8+T lymphocytes are prognostic factors of human ovarian cancer. Proceeding of the National Academy of Sciences (2007): 104: 3360-3365.
5. Thompson R H et al., Significance of B7-H1 overexpression in kidney cancer. Clinical genitourin Cancer (2006): 5: 206-211.
6. Nomi, T. Sho, M., Akahori, T., et al., Clinical significance and therapeutic potential of the programmed death-1 ligand/programmed death-1 pathway in human pancreatic cancer. Clinical Cancer Research (2007); 13: 2151-2157.
7. Ohigashi Y et al., Clinical significance of programmed death-1 ligand-1 and programmed death-1 ligand 2 expression in human esophageal cancer. Clin. Cancer Research (2005): 11: 2947-2953.
8. Inman et al., PD-L1 (B7-H1) expression by urothelial carcinoma of the bladder and BCG-induced granulomata: associations with localized stage progression. Cancer (2007): 109: 1499-1505.
9. Shimauchi T et al., Augmented expression of programmed death-1 in both neoplasmatic and nonneoplastic CD4+ T-cells in adult T-cell Leukemia/Lymphoma. Int. J. Cancer (2007): 121: 2585-2590.
10. Gao et al., Overexpression of PD-L1 significantly associates with tumor aggressiveness and postoperative recurrence in human hepatocellular carcinoma. Clinical Cancer Research (2009) 15: 971-979.
11. Nakanishi J., Overexpression of B7-H1 (PD-L1) significantly associates with tumor grade and postoperative prognosis in human urothelial cancers. Cancer Immunol Immunother. (2007) 56: 1173-1182.
12. Hino et al., Tumor cell expression of programmed cell death-1 is a prognostic factor for malignant melanoma. Cancer (2010): 00: 1-9.
13. Ghebeh H., Foxp3+ tregs and B7-H1+/PD-1+T lymphocytes co-infiltrate the tumor tissues of high-risk breast cancer patients: implication for immunotherapy. BMC Cancer. 2008 Feb. 23; 8:57.
14. Ahmadzadeh M. et al., Tumor antigen-specific CD8 T cells infiltrating the tumor express high levels of PD-1 and are functionally impaired. Blood (2009) 114: 1537-1544.
15. Thompson R H et al., PD-1 is expressed by tumor infiltrating cells and is associated with poor outcome for patients with renal carcinoma. Clinical Cancer Research (2007) 15: 1757-1761.
16. Knollman H. et al., Muscle-invasive urothelial bladder cancer: an update on systemic therapy. Ther Adv Urol (2015) 7(6): 312-330.
17. Zibelman M. et al., Checkpoint Inhibitors and Urothelial Carcinoma: The Translational Paradigm. Oncology (2016) 30(2): 160-162.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1: A method for treating an urothelial cancer in an individual comprising administering to the individual a combination therapy which comprises an antagonist of a Programmed Death 1 protein (PD-1) and eribulin or a pharmaceutically acceptable salt thereof.

2: The method of claim 1, wherein the individual is a human.

3: The method of claim 1, wherein the urothelial cancer is a solid tumor.

4: The method of claim 1, wherein the urothelial cancer is metastatic or a locally advanced urothelial cancer.

5: The method of claim 1, wherein the urothelial cancer is metastatic.

6: The method of claim 1, wherein the antagonist of PD-1 is an antibody or antigen-binding fragment thereof which specifically binds to PD-1.

7: The method of claim 6, wherein the antibody or antigen binding fragment thereof comprises three heavy chain CDRs (CDRH1, CDRH2, and CDRH3) and three light chain CDRs (CDRL1, CDRL2, and CDRL3), wherein:
a) CDRL1 comprises of the amino acid sequence set forth in SEQ ID NO: 7;
b) CDRL2 comprises of the amino acid sequence set forth in SEQ ID NO: 8;
c) CDRL3 comprises of the amino acid sequence set forth in SEQ ID NO: 9;
d) CDRH1 comprises of the amino acid sequence set forth in SEQ ID NO: 10;
e) CDRH2 comprises of the amino acid sequence set forth in SEQ ID NO: 11; and
CDRH3 comprises of the amino acid sequence set forth in SEQ ID NO: 12.

8: The method of claim 7, wherein the antibody or antigen binding fragment thereof, comprises a variable heavy region comprising an amino acid sequence as set forth in SEQ ID NO: 13 and a light chain variable region comprising an amino acid sequence as set forth in SEQ ID NO: 15.

9: The method of claim 1, wherein the PD-1 antagonist is pembrolizumab and the pharmaceutically acceptable salt of eribulin is eribulin mesylate.

10: A medicament comprising an antagonist of a Programmed Death 1 protein (PD-1) for use in combination with eribulin or a pharmaceutically acceptable salt thereof for treating an urothelial cancer in an individual.

11: A medicament comprising eribulin or a pharmaceutically acceptable salt thereof for use in combination with an antagonist of a Programmed Death 1 protein (PD-1) for treating an urothelial cancer in an individual.

12: The medicament of claim 10, wherein the individual is a human.

13: The medicament of claim 10, wherein the urothelial cancer is a solid tumor that tests positive for PD-L1 expression by an immunohistochemical (IHC) assay.

14: The medicament of claim 10, wherein the urothelial cancer is metastatic or a locally advanced urothelial cancer.

15: The medicament of claim 10, wherein the urothelial cancer is metastatic.

16: The medicament of claim 10, wherein the PD-1 antagonist is pembrolizumab and the pharmaceutically acceptable salt of eribulin is eribulin mesylate.

17: The medicament of claim 16, wherein pembrolizumab is formulated as a liquid medicament which comprises 25 mg/ml pembrolizumab, 7% (w/v) sucrose, 0.02% (w/v) polysorbate 80 in 10 mM histidine buffer pH 5.5, and eribulin or a pharmaceutically acceptable salt thereof is formulated as a liquid medicament which comprises 0.5 mg/mL eribulin mesylate or a pharmaceutically acceptable salt thereof.

18: A kit which comprises a first container, a second container and a package insert, wherein the first container comprises at least one dose of a medicament comprising an antagonist of a Programmed Death 1 protein (PD-1), the second container comprises at least one dose of a medicament comprising eribulin or a pharmaceutically acceptable salt thereof, and the package insert comprises instructions for treating an individual for an urothelial cancer using the medicaments.

19: The kit of claim 18, wherein the instructions state that the medicaments are intended for use in treating an individual having an urothelial cancer that tests positive for PD-L1 expression by an immunohistochemical (IHC) assay.

20: The kit of claim 18, wherein the individual is a human.

21: The kit of claim 18, wherein the PD-1 antagonist is pembrolizumab formulated as a liquid medicament which comprises 25 mg/ml pembrolizumab, 7% (w/v) sucrose, 0.02% (w/v) polysorbate 80 in 10 mM histidine buffer pH 5.5 and the pharmaceutically acceptable salt of eribulin is eribulin mesylate formulated as a liquid medicament which comprises 0.5 mg/mL eribulin mesylate.

22: The kit of claim 18, wherein the urothelial cancer is cancer metastatic urothelial cancer or a locally advanced urothelial cancer.

23: The kit of claim 18, wherein the urothelial cancer is metastatic.

24: A method for treating a human individual diagnosed with urothelial cancer, comprising administering to the individual a combination therapy which comprises pembrolizumab and eribulin or a pharmaceutically acceptable salt thereof, wherein the eribulin or the pharmaceutically acceptable salt thereof is administered at a dose of 1.4 mg/m², 1.1 mg/m², or 0.7 mg/m² on days 1 and 8 of a 21-day cycle and pembrolizumab is administered at a dose of 200 mg Q3 W.

25: A medicament comprising:
(a) pembrolizumab for use in combination with eribulin or a pharmaceutically acceptable salt thereof for treating an urothelial cancer in a human individual by a method comprising administering to the individual eribulin or a pharmaceutically acceptable salt thereof at a dose of 1.4 mg/m², 1.1 mg/m², or 0.7 mg/m² on days 1 and 8 of a 21-day cycle and pembrolizumab at a dose of 200 mg Q3W; or
(b) eribulin or a pharmaceutically acceptable salt thereof for use in combination with pembrolizumab for treating cancer urothelial cancer in a human individual by a method comprising administering to the individual eribulin or a pharmaceutically acceptable salt thereof at a dose of 1.4 mg/m², 1.1 mg/m², or 0.7 mg/m² on days 1 and 8 of a 21-day cycle and pembrolizumab at a dose of 200 mg Q3W.
